(19) United States
(12) Patent Application Publication
(10) Pub. No.: US 2011/0028794 A1
(43) Pub. Date: Feb. 3, 2011

(54) METHODS AND DEVICES FOR PROVIDING ACCESS INTO A BODY CAVITY

(75) Inventors: Christopher W. Widenhouse, Clarksville, OH (US); Jason R. Lesko, Harrison, OH (US); Michael V. Sherrill, Batavia, OH (US); Tamara S. V. Widenhouse, Clarksville, OH (US)

Correspondence Address: Ethicon Endo-Surgery/Nutter, McClennen & Fish LLP, Seaport West, 155 Seaport Blvd., Boston, MA, 02210-2604, US

(73) Assignee: Ethicon Endo-Surgery, Inc., Cincinnati, OH (US)

(21) Appl. No.: 12/512,568

(22) Filed: Jul. 30, 2009

(51) Int. Cl. A61B 1/32; A61F 11/00

(52) U.S. Cl. 600/208; 606/108

(57) ABSTRACT

Methods and devices are provided for providing surgical access into a body cavity. In one embodiment, a surgical access device is provided that includes a housing having multiple access ports for receiving surgical instruments, and a retractor removably coupled to the housing and having a working channel configured to extend into a body cavity. With the housing and retractor mated together, a portion of the housing through which instruments can be inserted can rotate relative to the retractor. The retractor can be positioned in tissue using an inserter tool configured to seat the retractor and to automatically release the retractor into position within the tissue. The retractor can be removed from the tissue using a cord coupled to the retractor.

### FIELD OF THE INVENTION

The present invention relates to methods and devices for providing surgical access into a body cavity.

### BACKGROUND OF THE INVENTION

Abdominal laparoscopic surgery gained popularity in the late 1980s, when benefits of laparoscopic removal of the gallbladder over traditional (open) operation became evident. Reduced postoperative recovery time, markedly decreased post-operative pain and wound infection, and improved cosmetic outcome are well established benefits of laparoscopic surgery, derived mainly from the ability of laparoscopic surgeons to perform an operation utilizing smaller incisions of the body cavity wall.

Laparoscopic procedures generally involve insufflation of the abdominal cavity with CO₂ gas to a pressure of around 15 mm Hg. The abdominal wall is pierced and a 5-10 mm in diameter straight tubular cannula or trocar sleeve is then inserted into the abdominal cavity. A laparoscopic telescope connected to an operating room monitor is used to visualize the operative field, and is placed through a the trocar sleeve. Laparoscopic instruments (graspers, dissectors, scissors, retractors, etc.) are placed through two or more additional trocar sleeves for the manipulations by the surgeon and surgical assistant(s).

Recently, so-called “mini-laparoscopy” has been introduced utilizing 2-3 mm diameter straight trocar sleeves and laparoscopic instruments. When successful, mini-laparoscopy allows further reduction of abdominal wall trauma and improved cosmesis. Instruments used for mini-laparoscopic procedures are, however, generally more expensive and fragile. Because of their performance limitations, due to their smaller diameter (weak suction-irrigation system, poor durability, decreased video quality), mini-laparoscopic instruments can generally be used only on selected patients with favorable anatomy (thin cavity wall, few adhesions, minimal inflammation, etc.). These patients represent a small percentage of patients requiring laparoscopic procedures. In addition, smaller 2-3 mm incisions may still cause undesirable cosmetic outcomes and wound complications (bleeding, infection, pain, keloid formation, etc.).

Since the benefits of smaller and fewer body cavity incisions are proven, it would be desirable to perform an operation utilizing only a single incision. An umbilicus is well-hidden and the thinnest and least vascularized area of the abdominal wall. The umbilicus is generally a preferred choice of abdominal cavity entry in laparoscopic procedures. An umbilical incision can be easily enlarged (in order to eviscerate a larger specimen) without significantly compromising cosmesis and without increasing the chances of wound complications.

Thus, there is a need for instruments and trocar systems which allow laparoscopic procedures to be performed entirely through the umbilicus or a surgical port located elsewhere while at the same time allowing adjustment of instrument position during the surgical procedure.

### SUMMARY OF THE INVENTION

The present invention generally provides methods and devices for providing surgical access into a body cavity. In one embodiment, a surgical access device is provided that includes an outer housing defining a working channel, an inner housing rotatably mated to the outer housing, and a retractor protector mated to the inner housing and rotatable with the inner housing relative to the outer housing. The inner housing has a plurality of sealing ports each configured to receive an instrument inserted therethrough and into the working channel.

The surgical access device can vary in any number of ways. The outer housing can have an insufflation port extending from a wall thereof, and the inner housing can be rotatable relative to the insufflation port. A locking ring can be removably mated to the outer housing in one of a plurality of predetermined positions, and a flexible elongate cannula can be removably mated to the locking ring. A cap can be releasably mated to one of the sealing ports to reduce a diameter of the one of the sealing ports. A flexible retractor can extend distally from the outer housing and have an opening extending therethrough for forming a pathway through tissue into a body cavity. The retractor protector can extend into the opening of the flexible retractor.

The retractor protector can have a variety of configurations. In one embodiment, the retractor protector can include an outer layer and an inner layer disposed within the outer layer. The outer and inner layers can each include a plurality of distally extending fingers that at least partially overlap and engage one another.

In another embodiment, a surgical access device is provided that includes a flexible retractor configured to be positioned in an opening in tissue, a retractor ring releasably mated to the flexible retractor, a locking ring releasably mated to the retractor ring in a fixed position, and an outer housing releasably mated to the locking ring in a fixed position. The outer housing has an inner housing rotatably disposed therein, and the inner housing has a plurality of sealing ports extending therethrough.

The surgical access device can have any number of variations. The flexible retractor can include a flexible elongate cannula with a flexible proximal ring member and a flexible distal ring member. The retractor ring can receive the flexible proximal ring member therein to releasably mate the retractor ring to the flexible retractor. The outer housing and the retractor ring can each include a beveled surface configured to engage a proximal portion of the flexible retractor therebetween. The locking ring can define a plurality of predetermined rotational orientations for releasably mating to the outer housing. The outer housing can releasably mate to the locking ring in a plurality of predetermined fixed positions. A depressible tab on the outer housing can have an engaged position for mating the outer housing to the locking ring in a fixed position, and can have a released position for allowing release of the outer housing from the locking ring. The outer housing can rotate relative to the locking ring to lock thereto and unlock therefrom.

In another aspect, a surgical method is provided that includes positioning a flexible retractor in an opening in tissue of a patient such that a proximal portion of the retractor is located outside the patient and a distal portion of the retractor is disposed in a body cavity underlying the tissue, mating a retractor ring to the proximal portion of the flexible retractor located outside the patient, mating a locking ring to the retractor ring, mating an outer housing to the locking ring, and inserting an instrument through one of a plurality of sealing ports in an inner housing rotatably disposed within the outer housing.

In some embodiments, mating the outer housing to the locking ring can include positioning an insufflation port on the outer housing in a desired rotational position. Alternatively or additionally, mating the outer housing to the locking ring can include locking the outer housing to the locking ring in one of a plurality of predetermined fixed positions.

The method can have any number of other variations. For example, the method can include manipulating the instrument to rotate the inner housing relative to the outer housing. For another example, inserting the instrument through one of the plurality of sealing ports in the inner housing can include inserting the instrument through a retractor protector mated to the inner housing and rotatable therewith. For yet another example, mating the retractor ring to the proximal portion of the flexible retractor can include positioning a flexible proximal ring of the flexible retractor within the retractor ring.

In yet another aspect, an inserter tool is provided that includes an elongate shaft having a proximal handle portion and a flexible distal portion. The flexible distal portion has opposed side rails that define a channel extending longitudinally through at least a portion of the flexible distal portion. The channel includes first and second longitudinally extending recesses configured to seat opposed portions of a flexible ring. The flexible distal portion includes first and second retention members extending from the opposed side rails and toward the channel such that the retention members are configured to prevent the flexible ring from being pulled laterally out of the channel.

The inserter tool can vary in any number of ways. The flexible distal portion can have a thickness that is less than a thickness of the proximal handle portion. In some embodiments, the elongate shaft can have indicia thereon configured to indicate an insertion depth of the elongate shaft through an opening in tissue and into a body cavity.

In still another aspect, a surgical kit is provided that includes a cannula having proximal and distal flexible annular rings, and a flexible sidewall extending between the proximal and distal flexible annular rings and defining an inner lumen extending through the cannula. The kit also include an inserter tool having a handle and a flexible elongate shaft extending from the handle. The flexible elongate shaft includes a channel extending longitudinally therethrough and configured to seat one of the proximal and distal flexible annular rings to retain the seated annular ring in a collapsed configuration. The kit can optionally include a proximal assembly configured to mate to the proximal flexible annular ring and including a plurality of sealing ports each configured to receive an instrument inserted therethrough and into the inner lumen extending through the cannula. The proximal assembly can have a variety of configurations. In some embodiments the proximal assembly can include a retractor ring releasably matable to the proximal flexible annular ring, a locking ring releasably matable to the retractor ring in a fixed position, and a housing releasably matable to the locking ring in a fixed position. The housing can also have a variety of configurations, such as including an outer housing releasably matable to the locking ring in a fixed position. The outer housing can have an inner housing rotatably disposed therein and having the plurality of sealing ports.

In another aspect, a surgical method is provided that includes positioning a distal annular ring on a cannula within a channel extending longitudinally through a flexible distal portion of an insertion tool such that the channel retains the distal annular ring in a collapsed configuration. The distal annular ring is coupled to a proximal annular ring by a flexible sidewall defining an inner lumen extending between the proximal and distal annular rings. The method also includes advancing the flexible distal portion of the insertion tool through tissue such that the proximal annular ring on the cannula abuts an outer surface of the tissue and causes the flexible distal portion to flex. The channel releases the distal annular ring when a predetermined force is applied to the channel. In some embodiments, at least one retention member on the flexible distal portion extending toward the channel can move away from the channel when the predetermined force is applied to the channel to release the distal annular ring from the channel.

The method can vary in any other number of ways. For example, the method can include attaching a proximal assembly to the proximal annular ring when the proximal annular ring on the cannula abuts the outer surface of the tissue. An instrument can be inserted through one of a plurality of sealing ports in the proximal assembly and through the inner lumen of the cannula to position a distal portion of the instrument in a body cavity underlying the tissue. For another example, the method can include pulling a cord extending through the inner lumen, around the distal annular ring, and between the flexible sidewall and the tissue to pull the distal annular ring through the tissue to remove the cannula from the tissue.

In another aspect, a surgical method is provided that includes implanting a cannula through an incision in tissue to position a proximal annular ring adjacent to an outer surface of the tissue and to position a distal annular ring adjacent to an inner surface of the tissue such that a flexible sidewall extending between the proximal and distal annular rings forms an opening through the tissue. The cannula has a cord extending through the opening, around the distal annular ring, and between the flexible sidewall and the tissue. The cord is pulled to pull the distal annular ring through the tissue to remove the cannula from the tissue. The method can have any number of variations.

### BRIEF DESCRIPTION OF THE DRAWINGS

The invention will be more fully understood from the following detailed description taken in conjunction with the accompanying drawings, in which:

FIG. 1 is a perspective view of one embodiment of a surgical access device;

FIG. 2 is an exploded view of the device of FIG. 1;

FIG. 3 is a side, cross-sectional view of the device of FIG. 1;

FIG. 4 is a top view of a housing of the device of FIG. 1;

FIG. 5 is a side, cross-sectional view of the housing of FIG. 4;

FIG. 6 is another side, cross-sectional view of the housing of FIG. 4;

FIG. 7 is an exploded view of the housing of FIG. 4;

FIG. 8 is a perspective view of a ring assembly and a retractor of the device of FIG. 1;

FIG. 9 is an exploded view of the ring assembly and the retractor of FIG. 8;

FIG. 10 is an exploded view of a lower portion of the housing of FIG. 4 and a locking ring of the ring assembly of FIG. 8;

FIG. 11 is a side view of the retractor of FIG. 8 with its distal flange being flexed;

FIG. 12 is a side view of the retractor of FIG. 11 with the distal flange being pushed proximally through an inner lumen of the retractor;

FIG. 13 is a side, partially cross-sectional view of the retractor of FIG. 12 being positioned by hand in an opening formed in tissue;

FIG. 14 is a side, partially cross-sectional view of the retractor of FIG. 8 positioned in an opening formed in the tissue and having its position therein being tactilely verified;

FIG. 15 is a perspective view of one embodiment of an inserter tool;

FIG. 16 is a top view of the inserter tool of FIG. 15;

FIG. 17 is a side, cross-sectional view of the inserter tool of FIG. 15;

FIG. 18 is a side view of the inserter tool of FIG. 15;

FIG. 19 is a cross-sectional view of a distal portion of the inserter tool of FIG. 15;

FIG. 20 is another cross-sectional view of a distal portion of the inserter tool of FIG. 15;

FIG. 21 is a perspective view of the retractor of FIG. 8 being attached to the inserter tool of FIG. 15;

FIG. 22 is a top, partial view of the retractor of FIG. 21 disposed in a channel of the inserter tool;

FIG. 23 is a side, partially cross-sectional view of the retractor of FIG. 22 mated to the inserter tool and being inserted in an opening formed in tissue;

FIG. 24 is a side, partially cross-sectional view of the retractor of FIG. 23 mated to the inserter tool and partially inserted in the opening formed in tissue;

FIG. 25 is a side, partially cross-sectional view of the retractor of FIG. 24 released from the inserter tool and positioned in the opening formed in tissue;

FIG. 26 is a perspective view of the ring assembly of FIG. 8 being attached to the retractor of FIG. 25 positioned in the opening formed in tissue;

FIG. 27 is a perspective view of the housing of FIG. 4 being attached to the ring assembly and the retractor of FIG. 26;

FIG. 28 is a side, partially cross-sectional view of the assembled housing, ring assembly, and retractor of FIG. 27 positioned in the opening formed in tissue and having a surgical instrument inserted therethrough;

FIG. 29 is a side, partially cross-sectional view of one embodiment of a cord being inserted through an opening formed in tissue;

FIG. 30 is a side view of the retractor of FIG. 8 attached to the inserter tool of FIG. 15 and being inserted into the tissue opening of FIG. 29 with the cord inserted therethrough;

FIG. 31 is a side view of the retractor and the inserter tool of FIG. 30 more deeply inserted into the tissue opening;

FIG. 32 is a side, partially-cross sectional view of the retractor of FIG. 31 positioned in the opening formed in tissue with the cord being positioned between the tissue and the retractor, around a distal end of the retractor, and through an inner lumen of the retractor;

FIG. 33 is a perspective view of the retractor of FIG. 8 mated to the inserter tool of FIG. 15 with one embodiment of a cord threaded through an inner lumen of the retractor;

FIG. 34 is a top view of the retractor of FIG. 32 with the ring assembly of FIG. 4 attached thereto and with the cord being manipulated to remove the retractor from the opening formed in tissue; and

FIG. 35 is a perspective view of the retractor and ring assembly of FIG. 33 with the cord being used to pull a distal end of the retractor through the retractor's inner lumen to remove the retractor from the opening formed in tissue.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Various exemplary methods and devices are provided for providing surgical access into a body cavity. In general, the methods and devices allow multiple surgical instruments to be inserted through independent access ports in a single surgical access device and into a body cavity. The instruments can be collectively rotatable about a central axis of the device, thus allowing for ease of manipulation within a patient's body. In one embodiment, a surgical access device includes a housing having multiple access ports or sealing ports for receiving surgical instruments, and a retractor removably coupled to the housing and having a working channel configured to extend into a body cavity. Each sealing port can include one or more sealing elements therein for sealing the port and/or forming a seal around a surgical instrument disposed therethrough. Mating features can be configured to align the housing and the retractor in a predetermined position relative to one another to allow for easy attachment and removal of the housing to and from the retractor. Once mated, a portion of the housing through which instruments can be inserted can rotate relative to the retractor, thereby helping to optimally position instruments inserted therethrough and into the body cavity in which the retractor extends. The device can also include at least one safety shield extending from the housing into the retractor to help protect the retractor from being damaged by instruments passed through the retractor's working channel.

The surgical access device can be positioned in an opening in tissue in any way to provide a working channel through the tissue to provide access to a body cavity underlying the tissue. In another general aspect, the methods and devices allow a surgical access device to be positioned in an opening in tissue with an inserter tool. The inserter tool can include an elongate shaft having a flexible distal end configured to releasably mate to at least a portion of the surgical access device. In one embodiment, the retractor of the surgical access device can be releasably coupled to the flexible distal end of the inserter tool. The inserter tool's distal end with the retractor attached thereto can be inserted through a tissue opening, with the inserter tool being configured to automatically release the retractor therefrom when the retractor extends through the tissue opening with opposed terminal ends of the retractor positioned on opposite sides of the tissue. The retractor can thereby be positioned easily, accurately, and hands-free in any sized tissue opening using a single, reusable surgical tool. The housing of the surgical access device can be mated to the retractor, either before, or in an exemplary embodiment, after the retractor is positioned in tissue. In some embodiments, the retractor can be removed from the tissue using a cord coupled to the retractor.

As indicated above, the various surgical access devices can include a wound protector, cannula, or other member for forming a pathway through tissue (hereinafter generally referred to as a retractor). The retractor can extend from the housing and it can be configured to be positioned within an opening in a patient's body, such as the umbilicus. The sealing ports can each define working channels extending through the housing and aligned with the retractor. Any and all of the surgical access devices described herein can also include various other features, such as one or more ventilation ports to allow evacuation of smoke during procedures that utilize cautery, and/or one or more insufflation ports through which the surgeon can insufflate the abdomen to cause pneumoperitenium, as described by way of non-limiting example in U.S. Patent Application No. 2006/0247673 entitled “Multi-port Laparoscopic Access Device” filed Nov. 2, 2006, which is hereby incorporated by reference in its entirety. The insufflation port can be located anywhere on the device, can have any size, and can accept a leur lock or a needle, as will be appreciated by those skilled in the art.

As discussed further below, any and all embodiments of a surgical access device can also include one or more retractor protectors or safety shields positioned through, in, and around any of the components and/or tissue to protect the components against puncture or tear by surgical instruments being inserted through the device. In addition, any and all embodiments of a surgical access device can include engagement and release mechanisms that allow certain components of the surgical access device to be removable as needed.

In use, and as also further discussed below, the surgical access devices disclosed herein can provide access to a patient's body cavity. The retractor can be positionable within an opening in a patient's body such that a distal portion of the retractor extends into a patient's body cavity and a proximal portion configured to couple to the housing is positioned adjacent to the patient's skin on an exterior of the patient's body. A lumen in the retractor can form a pathway through the opening in a patient's body so that surgical instruments can be inserted from outside the body to an interior body cavity. The elasticity of the skin of the patient can assist in the retention of the retractor in the body opening or incision made in the body. The retractor can be placed in any opening within a patient's body, whether a natural orifice or an opening made by an incision. In one embodiment, the retractor can be substantially flexible so that it can easily be maneuvered into and within tissue as needed. In other embodiments, the retractor can be substantially rigid or substantially semi-rigid. The retractor can be formed of any suitable material known in the art, e.g., silicone, urethane, thermoplastic elastomer, and rubber. Non-limiting examples of retractors include a Hakko® Wound Protector available from Hakko Medical Co. of Tokyo, Japan, an Alexis® Wound Protector available from Applied Medical Resources Corp. of Rancho Santa Margarita, Calif., and a Mobius® Retractor available from Apple Medical Corp. of Marlborough, Mass.

Typically, during surgical procedures in a body cavity, such as the abdomen, insufflation is provided through the surgical access device to expand the body cavity to facilitate the surgical procedure. Thus, in order to maintain insufflation within the body cavity, most surgical access devices include at least one seal disposed therein to prevent air and/or gas from escaping when surgical instruments are inserted therethrough. Various sealing elements are known in the art, but typically the surgical access device can include at least one instrument seal that forms a seal around an instrument disposed therethrough, but otherwise does not form a seal when no instrument is disposed therethrough, at least one channel seal or zero-closure seal that seals the working channel created by the sealing port when no instrument is disposed therethrough, or a combination instrument seal and channel seal that is effective to both form a seal around an instrument disposed therethrough and to form a seal in the working channel when no instrument is disposed therethrough. A person skilled in the art will appreciate that various seals known in the art can be used including, e.g., duckbill seals, cone seals, flapper valves, gel seals, diaphragm seals, lip seals, iris seals, etc. A person skilled in the art will also appreciate that any combination of seals can be included in any of the embodiments described herein, whether or not the seal combinations are specifically discussed in the corresponding description of a particular embodiment.

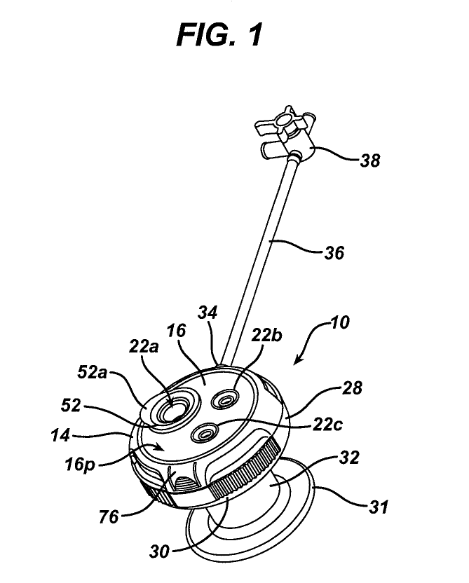

In an exemplary embodiment, shown in FIGS. 1-3, a surgical access device 10 is provided having a proximal portion 24 including a housing 12 and a ring assembly 26, and a distal portion 20 including a retractor 18 having an inner pathway, inner lumen, or working channel 18a extending therethrough. As shown in the illustrated embodiment, the housing 12 can be configured to have one or more surgical instruments inserted therethrough and can include an outer housing 14 and an inner housing 16 that defines at least one sealing or access port. While the inner housing 16 can define any number of sealing ports, in the illustrated embodiment, the inner housing 16 defines first, second, and third sealing ports 22a, 22b, 22c that extend through the inner housing 16 and that respectively seat first, second, and third sealing elements, as discussed further below. The housing 12 can be removably coupled to the ring assembly 26, which in the illustrated embodiment includes a locking ring 28 and a retractor ring 30 configured to releasably or fixedly mate to each other. The housing 12 can be configured to releasably mate to the locking ring 28, and the retractor ring 30 can be configured to releasably mate to the retractor 18. The retractor 18 can thus be configured to distally extend from the housing 12 and to provide a pathway through tissue into a body cavity. In the embodiment shown, the retractor 18 is flexible and has a proximal flange 29 and a distal flange 31 with an inner elongate portion 32 extending therebetween. The inner housing 16 can be movable with respect to the outer housing 14 and the retractor 18, as will be discussed in more detail below. Such a configuration can help facilitate instrument positioning in a body cavity to which the device 10 provides access.

The device 10 can also include an insufflation port 34 in the outer housing 14, although a person skilled in the art will appreciate that the insufflation port 34 can be located elsewhere in the housing 12 or in other locations. A person skilled in the art will also appreciate that the insufflation port 34 can have a variety of configurations. Generally, the insufflation port 34 can be configured to pass an insufflation fluid through a flexible insufflation tube 36 and into an insufflation orifice of the insufflation port 34 where the fluid can flow between the outer housing 14 and the inner housing 16, into the retractor's pathway 18a, and into a body cavity. A stopcock 38 can control fluid flow through the insufflation tube 36. As discussed further below, by having the insufflation port 34 extend from the outer housing 14, the insufflation port 34 can be configured to have a fixed rotational orientation relative to the retractor 18 regardless of the rotational orientation of the inner housing 16 relative to the outer housing 14, thereby reducing chances of the tube 36 twisting and/or becoming blocked or severed when the inner housing 16 rotates. In this way, the insufflation port 34 and the tube 36 extending therefrom can be selectively positioned at a location less likely to cause interference with surgical instruments and/or surgical staff during a surgical procedure.

The housing 12 of the surgical access device 10 can have a variety of configurations. As shown in this embodiment, the outer housing 14 can be configured to rotatably seat the inner housing 16, and the ring assembly 26 can be in the form of two rings, e.g., the locking ring 28 and the retractor ring 30, configured to be disposed between the housing 12 and the retractor 18 to releasably mate and form a seat and seal between the housing 12 and a distal portion of the device 10, e.g., the retractor 18. The retractor 18, the inner housing 16, the outer housing 14, and the ring assembly 26 can each have various sizes, shapes, and configurations, as discussed further below.

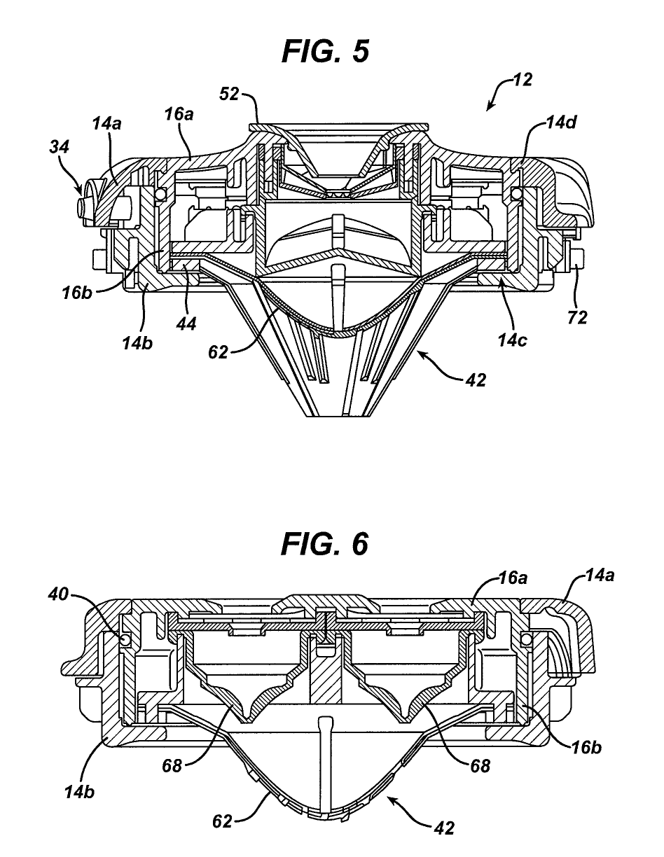

As noted above, the retractor 18 can extend distally from the proximal portion 24 of the device 10, and it can be configured to be positioned in an opening formed in tissue. The inner elongate portion 32 of the retractor 18 can have a diameter less than a diameter of the proximal and distal flanges 29, 31, which can have the same diameter or different diameters from one another. The proximal flange 29 can be configured to be seated within the retractor ring 30, as shown in FIGS. 4 and 5, and optionally attached thereto using an adhesive, sealant, complementary threads, or any other attachment mechanism, as will be appreciated by a person skilled in the art. A proximal o-ring 29a can be optionally positioned within the proximal flange 29 to help provide structural support to the retractor 18 within the retractor ring 30. A distal o-ring 31 a can optionally be positioned within the distal flange 31 to provide structural support to the retractor 18 within a patient's body. The proximal and distal o-rings 29a, 31a in this embodiment are substantially flexible, but one or both can be substantially flexible or substantially rigid as needed for use in a particular application. The retractor 18 can be molded as a cylindrical tube having a substantially constant diameter. The proximal and distal flanges 29, 31 can be formed by stretching proximal and distal ends of the cylindrical tube respectively around the proximal and distal o-rings 29a, 31a to form the proximal and distal flanges 29, 31 having larger diameters than the inner elongate portion 32. A person skilled in the art will appreciate that the o-rings 29a, 31a can be positioned in the proximal and distal flanges 29, 31 in any way, such as by stretching and folding ends of the molded retractor around the o-rings 29a, 31a and bonding the edges around the o-rings 29a, 31a.

The housing 12 can include, as illustrated in FIGS. 1-7, the outer housing 14 having upper and lower portions 14a, 14b fixedly coupled together to define a working channel extending therethrough and to form a substantially rigid cylindrical or circular member. The inner housing 16 can also include upper and lower portions 16a, 16b fixedly coupled together to form a substantially rigid cylindrical or circular member. The inner housing 16 can be movably seated in the outer housing's working channel, as discussed further below. One or more seal members can be positioned between the outer and inner housings 14, 16 to help form a seat and seal therebetween. In the illustrated embodiment, a proximal o-ring 40 is positioned between the upper portions 14a, 16a of the outer and inner housings 14, 16. The proximal o-ring 40 can be positioned proximal to the insufflation port 34 to allow fluid to flow distally between the outer and inner housings 14, 16 and into the retractor 18 and a body of a patient. A retractor protector 42 and a retractor protector retainer 44 in the illustrated embodiment are positioned between the lower portions 14b, 16b of the outer and inner housings 14, 16.

As surgical instruments are inserted through the surgical access device embodiments described herein, a risk can exist that a particularly sharp instrument may tear or puncture a portion of the retractor or nearby tissue. Accordingly, in any and all of the embodiments described herein, a retractor protector or safety shield can optionally be included to reduce the risk of tearing or puncture by a surgical instrument. In general the retractor protector can be of a material that is relatively smooth and with a low coefficient of friction to allow ease of passage of instruments, but resistant to tearing and puncture. For example, the retractor protector can be formed of silicone, urethane, thermoplastic elastomer, rubber, polyolefins, polyesters, nylons, fluoropolymers, and any other suitable materials known in the art. The retractor protector can generally provide a liner for a retractor or tissue and can be detachable from a surgical access device so it can be used as needed in a particular procedure.

In the illustrated embodiment, the device 10 includes the retractor protector 42 as part of the proximal portion 24 of the device 10 that is releasably matable to the device's distal portion 20, although a retractor protector 42 can be part of the proximal portion 24 or the distal portion 20 of the device 10. The retractor protector 42 can be configured to extend at least partially into the inner lumen 18a of the retractor 18 to thereby provide a protective lining as surgical instruments are inserted through the device 10. The retractor protector 42 can have a length corresponding to a length of the retractor 18, but can also have a length less than (as shown) or considerably longer than the length of the retractor 18 depending on a specific application. The retractor protector 42 can be mated to the device's proximal portion 24, e.g., the housing 12, using any attachment mechanism, e.g., adhesive, screws, press fit, etc., as will be appreciated by a person skilled in the art. As illustrated, the retractor protector 42 can be configured to be held by press fit between a proximal surface 44a of the retractor protector retainer 44 and a distal surface of the lower portion 16b of the inner housing 16. A distal surface 44b of the retractor protector retainer 44 can slidably engage an inner radially-inward extending circumferential lip 14c of the outer housing's lower portion 14b. The retractor protector 42 can thereby move with the inner housing 16 relative to the outer housing 14 and the retractor 18, as discussed further below.

The retractor protector 42 can have any size, shape, and configuration. In the illustrated embodiment, the retractor protector 42 includes a circumferentially expandable, cone-shaped member having an outer layer 42a and an inner layer 42b configured to be disposed within the outer layer 42a. The outer and inner layers 42a, 42b can each respectively include a continuous circumferential proximal rim 46a, 46b extending radially outward as shown in this embodiment, or one or both can have a plurality of flanges extending radially outward therefrom. The outer and inner layers 42a, 42b can include any number of flanges, and the flanges can be spaced equidistantly or any other distance apart from one another around their respective proximal rims 46a, 46b. The outer and inner flanges can each be configured to at least partially overlap to form a continuous proximal flange of the retractor protector 42. Alternatively, a portion of the outer and inner flanges can be configured to engage one another to form a “broken” proximal flange of the retractor protector 42. In other embodiments, none of the outer and inner flanges can overlap one another when the inner layer 42b is disposed in the outer layer 42a.

The outer and inner layers 42a, 42b of the retractor protector 42 can also include a plurality of respective distal elongate fingers 48a, 48b distally extending from the proximal rims 46a, 46b and configured to at least partially overlap and engage one another when the inner layer 42b is disposed in the outer layer 42a to form a continuous distal surface to help protect an entire circumference of an inner wall of the inner elongate portion 32 of the retractor 18. The distal fingers 48a, 48b can thus be configured to protect the inner elongate portion 32 of the retractor 18 from damage, but they can be configured to be selectively movable when in contact with a surgical instrument such that the surgical instrument can optionally push between the distal fingers 48a, 48b to help provide the surgical instrument with free angular range of motion through the device 10. The distal fingers 48a, 48b can also be configured to be selectively movable when the retractor 18 bends when in position in tissue, if the retractor 18 is flexible. The retractor protector 42 can include any number of distal fingers 48a, 48b, e.g., six outer and six inner fingers 48a, 48b.

A retractor protector can include a plurality of layers as discussed above, or a retractor protector can be a singular member, which can make the retractor protector easier to dispose in a retractor. Exemplary singular member retractor protectors are described in more detail in U.S. patent application Ser. No. 12/399,625 entitled “Methods And Devices For Providing Access Into A Body Cavity,” filed on Mar. 6, 2009, which is hereby incorporated by reference in its entirety. Exemplary embodiments of various safety shields are described in more detail in previously mentioned U.S. Patent Application No. 2006/0247673 entitled “Multi-port Laparoscopic Access Device” filed Nov. 2, 2006 and in U.S. application Ser. No. 12/399,482 entitled “Methods and Devices for Providing Access to a Body Cavity” filed on Mar. 6, 2009 and in U.S. application Ser. No. 12/242,765 entitled “Surgical Access Device” filed on Sep. 30, 2008, which are hereby incorporated by reference in their entireties.

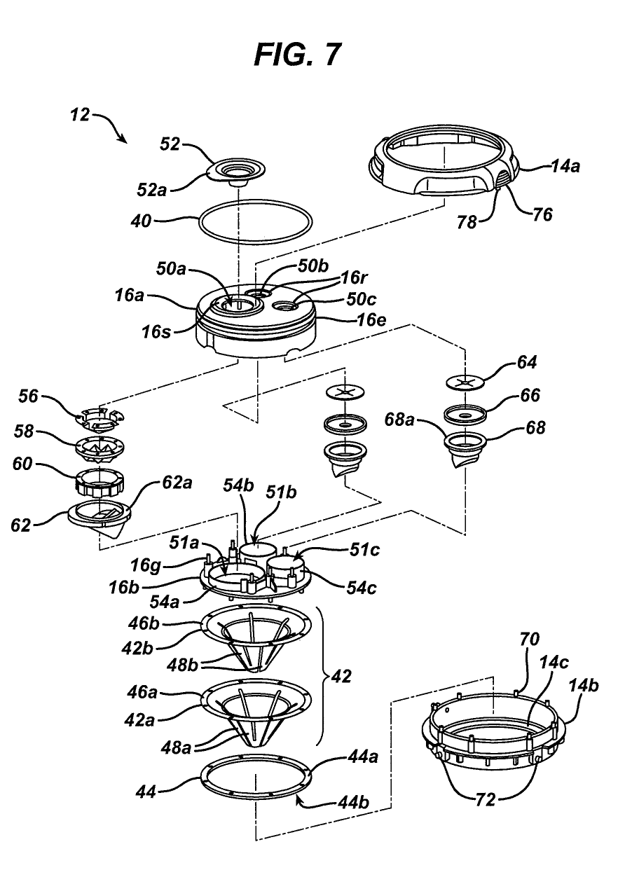

The inner housing 16 can have a variety of sizes, shapes, and configurations, as can the sealing ports 22a, 22b, 22c formed therein. As shown in FIG. 7, the sealing ports 22a, 22b, 22c can be defined by cut-outs or openings 50a, 50b, 50c in the upper portion 16a of the inner housing 16 and corresponding cut-outs or openings 51a, 51b, 51c in the lower portion 16b of the inner housing 16. The openings 50a, 50b, 50c can extend through a proximal surface 16p of the inner housing's upper portion 16a such that they are exposed within the working channel defined by the outer housing 14, and the openings 51a, 51b, 51c in the lower portion 16b can be aligned with the openings 50a, 50b, 50c in the upper portion 16a such that surgical instruments can be inserted into the openings 50a, 50b, 50c, 51a, 51b, 51c and into the retractor 18. A person skilled in the art will appreciate that there can be any number of sealing ports formed in the inner housing 16 that can be arranged in any way in the inner housing 16. As shown in the illustrated embodiment, each of the sealing ports 22a, 22b, 22c can share a central axis that extends substantially perpendicular to a plane of the proximal surface 16p of the inner housing 16 and that is substantially parallel to a longitudinal axis of the retractor 18. The sealing ports 22a, 22b, 22c can each be in a fixed position relative to the inner housing 16, but any one or more components in each sealing port can be angled relative to the inner housing 16 and/or rotatable or otherwise movable relative to the inner housing 16 and/or other portion(s) of the housing 12. The sealing ports 22a, 22b, 22c can be radially arranged around a central axis or center-point of the housing 12, e.g., a central axis or center-point of the inner housing 16, such that each of the sealing ports 22a. 22b, 22c can have a central axis that differs from central axes of the other sealing ports 22a, 22b, 22c. Each of the sealing ports 22a, 22b, 22c can have central axes located any distance from the center point of the housing 12. In an exemplary embodiment, to optimize instrument movement, the central axes can be located about 1.015 in. (2.58 cm) from the housing's center point for the larger port 22a and about 0.9 in (2.29 cm) from the housing's center point for the smaller ports 22b, 22c.

The upper openings 50a, 50b, 50c and the lower openings 51a, 51b, 51c can also have any combination of sizes and shapes. As shown, the port openings can each have a shape corresponding to a shape of the sealing element seated therein, which in the illustrated embodiment is substantially circular as will be discussed further below. In an exemplary embodiment, the first port opening has a first diameter D1 that is larger than a second diameter D2 of the second and third port openings. For non-limiting example, the housing 12 can have a diameter of about 75 mm, the second diameter D2 can be in a range of about 6.0 to 7.5 mm, e.g., about 6.2 mm, and the first diameter D1 can be about 15.9 mm, e.g., about three times the second diameter D2.

The device 10 can optionally include at least one reducer cap selectively and removably matable to any of the sealing ports 22a, 22b, 22c to reduce a diameter thereof to allow a smaller surgical instrument to be inserted centrally therethrough while maintaining channel and instrument seals. The reducer cap can be mated to a sealing port in any way, such as by snap-fit. The reducer cap 52 can have a variety of shapes, sizes, and configurations. As shown in this embodiment, a reducer cap 52 can be pre-attached and removably matable to the larger, first port opening 22a to reduce the first diameter D1, e.g., from about 15 mm to about 5 mm. Also as shown in this embodiment, the reducer cap 52 can have a substantially circular proximal portion having a distal conical portion extending therefrom with an inner lumen extending through the proximal and distal portions. The reducer cap's inner lumen can be in communication with a working channel of a sealing port to which it is mated such that a surgical instrument can be inserted through the reducer cap 52 and into the sealing element seated in its associated sealing port. The reducer cap 52 can include a hand-hold, e.g., a tab 52a extending radially-outward in the proximal portion of the reducer cap 52, to help ease attachment and removal of the reducer cap 52 with the inner housing 16. In some embodiments, the reducer cap 52 can be removably matable to a sealing element disposed in a sealing port in addition to or instead of removably mating to the sealing port.

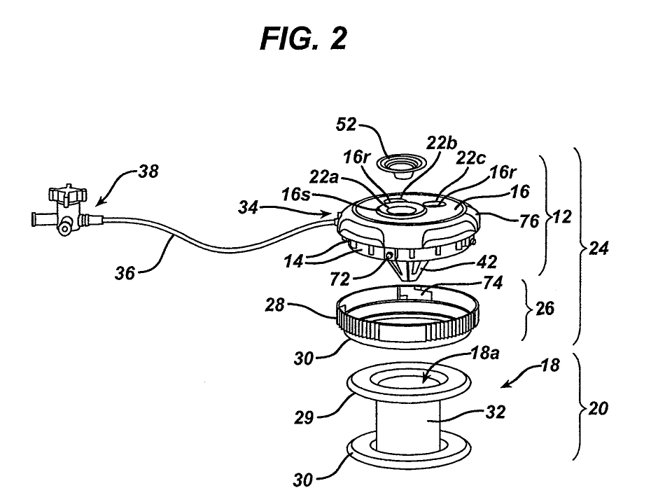

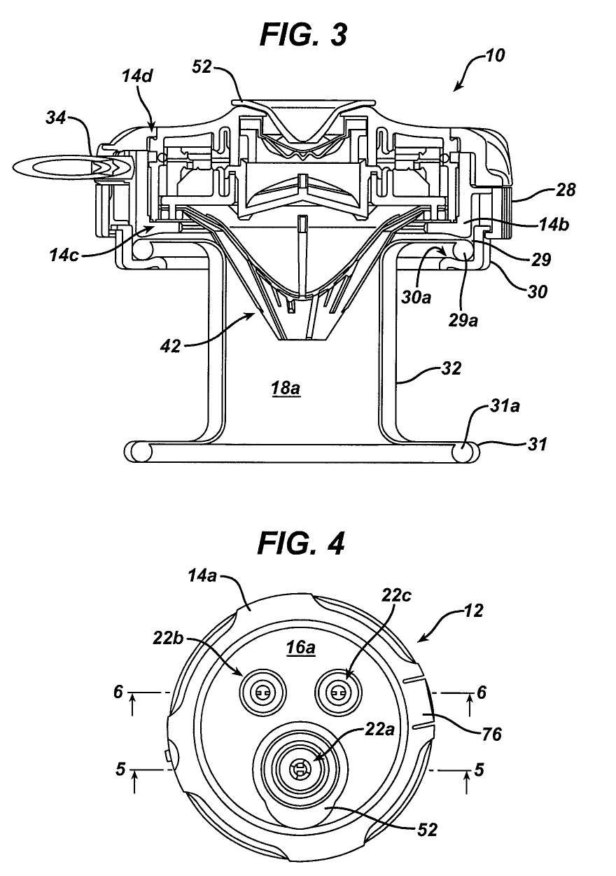

In some embodiments, the proximal surface 16p of the upper portion 16a of the inner housing 16 can be substantially flat with the port openings 50a, 50b, 50c being formed in a same plane with each other, either co-planar parallel to the proximal surface 16p or recessed in the inner housing 16. Moreover, as shown in the embodiment in FIGS. 2 and 7, the proximal surface 16p of the upper portion 16a of the inner housing 16 can be non-planar with at least one recessed portion 16r extending in a plane distally displaced from and substantially parallel to a plane of the proximal surface 16p, and/or at least one raised portion 16s proximally displaced from and substantially parallel to a plane of the proximal surface 16p. The inner housing's one or more recessed portions and one or more raised portions can help compensate for sealing elements of different lengths to help prevent distal seal element openings of each of the sealing elements from contacting an interior of the retractor 18, at least when the surgical access device 10 is in a default position, e.g., as illustrated in FIGS. 1 and 3, and at least when the device 10 is not positioned in tissue and has no surgical instruments inserted therethrough.

In the illustrated embodiment, the lower portion 16b of the inner housing 16 has raised or proximally extending housings 54a, 54b, 54c that define the first, second, and third openings 51a, 51b, 51c in the inner housing's lower portion 16b. The second and third raised housings 54b, 54c have a larger height than the first raised housing 54a, but the raised housings 54a, 54b, 54c can have any height, same or different from any other raised housings, configured to help provide clearance room for the sealing elements seated therein to help prevent the sealing elements from contacting the retractor 18, as discussed below, at least when the surgical access device 10 is in the default position.

The sealing elements disposed in each sealing port 22a, 22b, 22c can be attached or mated to the inner housing 16 using any attachment or mating mechanism known in the art, but in the illustrated embodiment the sealing elements are engaged by an interference fit between the upper and lower portions 16a, 16b of the inner housing 16. In general, each of the sealing ports 22a, 22b, 22c can include an instrument seal and a channel or zero-closure seal disposed therein.

The sealing elements can have a variety of sizes, shapes, and configurations. As shown in the illustrated embodiment in FIG. 7, the first sealing element includes a fan seal 58, a fan seal protector 56 positioned concentric with and proximal to the fan seal 58, and a bottom ring 60 in which the fan seal 58 can be concentrically seated with the fan seal protector 56 to together form the instrument seal in the larger sealing port 22a. A distal duckbill seal 62 can be positioned concentric and distal to the bottom ring 60, and thus the bottom ring 60 can act as a spacer to separate the fan seal 58 and the duckbill seal 62. The duckbill seal 62 forms the channel or zero-closure seal to seal a working channel of the larger sealing port 22a when no instrument is disposed therethrough to prevent leakage of insufflation gases delivered through the surgical access device 10 to a body cavity. The duckbill seal 62 will generally not form a seal around an instrument inserted therethrough. In use, when a surgical instrument is passed through the larger sealing port 22a through a center opening of the fan seal protector 56 and the fan seal 58, the fan seal 58 can engage and form a seal around an outer surface of the instrument to thereby prevent the passage of fluids and gas through the seal. When no instrument is disposed therethrough, the center opening of the fan seal protector 56 and the fan seal 58 will generally not form a seal in the working channel of the larger sealing port 22a. A person skilled in the art will appreciate that while instrument seals in the form of fan seals are shown, any seal can be used and can be aligned in any way relative to the inner housing 16. Exemplary instrument seal configurations are described in more detail in U.S. Patent Publication No. 2004/0230161 entitled “Trocar Seal Assembly,” filed on Mar. 31, 2004, and U.S. patent application Ser. No. 10/687,502 entitled “Conical Trocar Seal,” filed on Oct. 15, 2003, which are hereby incorporated by reference in their entireties. When the instrument is further inserted through the duckbill seal 62, the instrument can open the duckbill seal 62 and pass into the working channel 18a of the retractor 18 when the retractor 18 is coupled to the housing 12.

As mentioned above, the larger sealing element can have any size, but in an exemplary embodiment, it can be sized for seating in the inner housing's first port opening having a diameter D1 of about 15.9 mm and for receiving instruments having shaft diameters in a range of about 4.7 to 15.7 mm. The fan seal 58 can have an inner septum having a diameter of less than about 4.7 mm, and the fan seal protector 56 can have an inner diameter greater than about 5.9 mm. These inner diameters of the fan seal 58 and the fan seal protector 56 can help protect against seal damage when a relatively large instrument, e.g., having a shaft diameter in a range of about 10 to 12 mm, is inserted therethrough while also preventing floating and allowing the fan seal 58 to accommodate insertion of a smaller instrument, e.g., having a shaft diameter of about 5 mm, when the reducer cap 52 is attached to the first port opening 22a.

The smaller sealing elements in the smaller sealing ports 22b, 22c can each include a distal duckbill seal 68 that provides a channel seal, and a proximal septum seal 66 that provides an instrument seal. A protective member 64 can be positioned proximal to the septum seal 66 to protect the septum seal 66 from accidental puncture. The septum seal 66 can optionally include a beveled edge on an interior circumference thereof, which can help facilitate instrument insertion therethrough. If the septum seal 66 has an interior beveled edge, the protective member 64 can have an inner diameter substantially equal to an outer diameter of the beveled circumferential edge, which can help protect the septum seal 66 without floating and without substantially limiting angular movement of instruments inserted therethrough. The smaller sealing ports 22a, 22b can generally be used in a manner similar to the larger sealing port 22a, with an instrument being insertable through a center opening in the protective member 64 and the septum seal 66 and then through the duckbill seal 68 and into a working channel of the retractor 18 when the retractor 18 is coupled to the housing 12. Although the second and third sealing elements are configured similar to each other in this embodiment, a person skilled in the art will appreciate that the second and third sealing elements can be configured different from one another.

Each of the distal duckbill seals 62, 68, the fan seal 58, the fan seal protector 56, and the septum seal 66 can include a radially-outward extending proximal flange 62a, 68a, 58a, 56a, 66a. The proximal flanges 62a, 68a, 58a, 56a, 66a can each be captured between a proximal surface of one of the raised housings 54a, 54b, 54c and an inner distal cylindrical rib or projection formed around each of the openings 50a, 50b, 50c in the upper portion 16a of the inner housing 16, thereby seating the sealing elements within their respective port openings in the inner housing 16. As noted above, however, the larger sealing port 22a includes a bottom ring 60 and the smaller sealing ports 22b, 22c include the protective member 64 that are also captured between the upper and lower portions 16a, 16b of the lower housing 16. The upper and lower portions 16a, 16b of the inner housing 16 can be sealingly engaged, thereby forming a seal around the sealing ports 22a, 22b, 22c. To seal together, one or more projections, e.g., cylindrical pegs or prongs 16g (see FIG. 7), can proximally extend from an inner surface of the inner housing's lower portion 16b and each be inserted into a corresponding cavity (not shown), e.g., a cylindrical bore, formed in an inner surface of the inner housing's upper portion 16a.

As mentioned above, the smaller sealing elements can each have any size, but in an exemplary embodiment, they can each be sized for seating in the inner housing's smaller port openings having diameters D2 in a range of about 6.0 to 7.5 mm and for receiving instruments having shaft diameters in a range of about 4.7 to 5.9 mm. The septum seal 66 can have an inner diameter in a range of about 1.9 to 3.4 mm, e.g., about 3.2 mm.

A person skilled in the art will appreciate that while channel or zero-closure seals in the form of duckbill seals are shown for the distal seals 62, 68, any seal, e.g., duckbill seals, cone seals, flapper valves, gel seals, diaphragm seals, lip seals, iris seals, non-linear sealing elements such sealing elements with an S-shaped opening, etc., same or different from any other of the other distal seals 62, 68 can be used and can be aligned in any way relative to the inner housing 16. Generally, a zero-closure seal can be configured to form a seal in a working channel when no instrument is disposed therethrough to thus prevent the leakage of insufflation gases delivered through the surgical access device to the body cavity. A duckbill seal can generally have opposed flaps that extend at an angle toward one another in a distal direction and that come together at a distal end to form a seal face. The opposed flaps can be movable relative to one another to allow the seal face to move between a closed position, in which no instrument is disposed therethrough and the seal face seals the working channel of the surgical access device, and an open position in which an instrument is disposed therethrough. A duckbill seal can include various other features, as described in more detail in U.S. application Ser. No. 11/771,263, entitled “Duckbill Seal with Fluid Drainage Feature,” filed on Jun. 29, 2007, which is hereby incorporated by reference in its entirety. In addition, the seal face of the duckbill seal can be in any nonlinear shape or configuration known in the art, for example in an S-shaped configuration, as described in more detail in U.S. Pat. No. 5,330,437, entitled “Self Sealing Flexible Elastomeric Valve and Trocar Assembly for Incorporating Same,” filed Nov. 12, 1993, which is hereby incorporated by reference in its entirety.

As mentioned above, the sealing ports 22a, 22b, 22c can be configured to be in a fixed position relative to the inner housing 16 and to rotate with the inner housing 16 relative to the outer housing 14 and the retractor 18, as discussed further below. However, any one or more of the sealing ports 22a, 22b, 22c can be configured to be movable relative to any one or more portions of the housing 12, such as the inner housing 16, the outer housing 14, or any others of the sealing ports 22a, 22b, 22c.

Also as mentioned above and as shown in FIGS. 5-7, the outer housing 14 can be configured as a substantially rigid cylindrical or circular member having the inner housing 16 disposed therein. Although the outer housing 14 can be a singular member or can have multiple portions mated together in any way, in the illustrated embodiment, pins 70 proximally extending from an outer perimeter of the outer housing's lower portion 14b can extend into corresponding bores (not shown) formed in a circumferential wall of the outer housing's upper portion 14a to mate the upper and lower portions 14a, 14b together. The circumferential wall of the outer housing's upper portion 14a and/or the inner housing's upper portion 16a can optionally include one or more cut-out portions (not shown) formed therein adjacent to a sealing port 22a, 22b, 22c that are configured to help angle surgical instruments inserted through the sealing ports 22a, 22b, 22c.

The inner housing 16 can be disposed and captured within the outer housing 14 in a variety of ways. As in the illustrated embodiment (see FIGS. 3, 5, and 7), the outer housing 14 can include complementary rotational surfaces configured to engage and allow rotation of the inner housing 16. A proximal surface of the inner radially-inward extending circumferential lip 14c formed on the outer housing's lower portion 14b can form a distal rotational surface that seats the inner housing 16, while a distal surface of an inner radially-inward extending circumferential lip 14d formed the outer housing's upper portion 14a can be a proximal rotational surface for the inner housing 16. As shown, the inner housing 16, e.g., the upper portion 16a, can include a circumferential groove 16e formed therein configured to receive the proximal lip 14d of the outer housing 14. A distal surface of the inner housing 16, e.g., a distal surface of the lower portion 16b, can be configured to rest on the distal lip 14c of the outer housing 14, which can also engage the retractor protector retainer 44 as discussed above. In this way, the inner housing 16 can rotate relative to the outer housing 14 by sliding along the inner lips 14c, 14d of the outer housing 14. The outer housing's inner lips 14c, 14d can also help prevent the inner housing 16 from tilting when rotating to help prevent unpredictable movement of one or more instruments inserted through the inner housing 16.

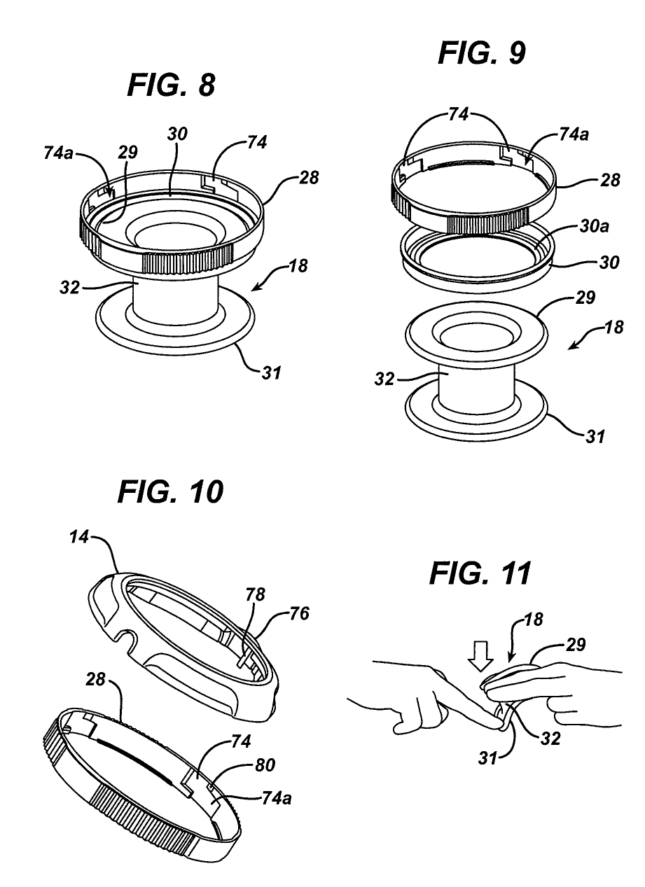

As indicated above, the ring assembly 26 can be positioned between the housing 12 and the retractor 18. Although the ring assembly 26 can have a variety of sizes, shapes, and configurations, and can include an integral, single ring, the ring assembly 26 can, as shown in FIGS. 1-3, 8, and 9, be in the form of two substantially circular rings 28, 30 releasably mated together in a fixed position.

In any and all of the surgical access device embodiments disclosed herein, an engagement and/or release mechanism can be included to allow the housing 12 to be separated from the ring assembly 26, to allow the housing 12 to be separated from the retractor 18, and/or to allow a sealing element to be removed from the inner housing 16. Any engagement and release mechanism known in the art, e.g., a snap-lock mechanism, corresponding threads, etc., can be used to releasably mate two components of the device 10. In one embodiment, the engagement and release mechanism can include a latch mechanism, as described by way of non-limiting example in U.S. application Ser. No. 12/242,765 entitled “Surgical Access Device” filed on Sep. 30, 2008, which is hereby incorporated by reference in its entirety.

As illustrated in the embodiment shown in FIGS. 1-10, the device 10 can include an engagement and release mechanism in the form of a bayonet latch mechanism. At least one bayonet foot or pin, e.g., four radially arranged bayonet feet or pins 72 spaced equidistantly or any other distance apart, can extend any length from an outer circumference of the housing 12, e.g., from an outer sidewall of the lower portion 16b of the outer housing 16, and they can be configured to engage corresponding slots 74 formed in an inner circumferential surface of the locking ring 28. The slots 74 can have any shape and size and can be the same as or different from any other of the slots 74. As discussed further below, the slots 74 can each include a vertically-extending portion in which the pins 72 can be proximally inserted and a laterally-extending portion in which the pins 72 can laterally slide. The bayonet pins 72 can have any shape and size and can be the same as or different from any other of the pins 72. The bayonet pins 72 on the outer housing 16 can be configured to be lowered into the vertically-extending portion of the slots 74 in the locking ring 28. Because the pins 72 can be properly aligned for insertion into the slots 74 in a predetermined number of rotational orientations, e.g., four predetermined orientations about 45° apart from each other as in the illustrated embodiment, the outer housing 16 can be attached to the locking ring 28 in a predictable orientation. Such predictability can allow the insufflation port 34 to be positioned in a desirable location. The bayonet pins 72 can be configured, as shown, to be identical and interchangeably-.lowered into any of the slots 74 in the locking ring 28. In some embodiments, any one or more of the bayonet pins 72 can differ from one another, and one or more of the slots 74 can correspondingly differ, such that the outer housing 14 can be configured to mate to the locking ring 28 in one or more predetermined rotational orientations, e.g., with different circumferentially arranged bayonet pins aligned with their corresponding different circumferentially arranged slots. In an embodiment where each of the bayonet pins 72 differs from one another and each of the slots 74 correspondingly differs from one another, the outer housing 14 can only be positioned in one predetermined rotational orientation relative to the locking ring 28 where the bayonet pins can each be simultaneously lowered into the corresponding slots. Embodiments of differing bayonet pins are described in more detail in previously mentioned U.S. application Ser. No. 12/399,482 entitled “Methods and Devices for Providing Access to a Body Cavity” filed on Mar. 6, 2009.

With the bayonet pins 72 engaging their corresponding slots 74, the outer housing 16, with the inner housing 14 and the retractor protector 42 coupled thereto, can then be rotated in a first direction, e.g., a clockwise direction, relative to the locking ring 28, thereby causing the bayonet pins 72 to travel laterally within the slots 74, e.g., within the laterally-extending portion of the slots 74, to a position in which the pins 72 abut terminal ends 74a of the slots 74, thereby locking the outer housing 16 to the locking ring 28. One or more of the slots 74 can angle proximally or distally (not shown) at their respective terminal ends 74a such that the bayonet pins 72 can proximally or distally slide and snap into the terminal ends 74a to help ensure that the bayonet pins 72 fully slide through the slots 74 to lock the housing 12 to the locking ring 28. In the illustrated embodiment, the bayonet pins 72 can only move in one, predictable direction, e.g., clockwise, to lock the outer housing 16 to the locking ring 28 because the laterally-extending portions of the slots 74 extend in that one, predictable direction from the vertically-extending portions of the slots 74. In some embodiments, the laterally-extending portions of the slots 74 can additionally or alternatively extend in a counter clockwise direction. Before being attached to the locking ring 28, the outer housing 16 can be pre-attached to a remainder of the housing 12, and the locking ring 28 can be pre-attached to the retractor ring 30 and the retractor 18, thereby allowing the device 10 to be fully assembled upon locking the outer housing 16 to the locking ring 28. The housing 12, e.g., an outer surface of the outer housing 14, can optionally include surface features, e.g., ridges, bumps, textured surface, etc., to help facilitate gripping and turning of the housing 12.

The outer housing 14 can optionally include a selectively engageable locking mechanism configured to alternatively allow locking of the outer housing 14 and the locking ring 28 and allow removal of the outer housing 14 from the locking ring 28. Although the locking mechanism can have a variety of configurations as will be appreciated by a person skilled in the art, in the illustrated embodiment the locking mechanism includes a depressible tab 76 on the outer housing 14. The tab 76 can have an engaged position for mating the outer housing 14 to the locking ring 28 in a fixed position such that the outer housing 14 cannot rotate, and have a released position for allowing rotation of the outer housing 14 and release of the outer housing 14 from the locking ring 28. The tab 76 can be configured to automatically be in the engaged position when the pins 72 reach the terminal ends 74a of the slots 74, and can be configured to be selectively movable from the engaged position to the released position by pressing and holding the tab 76 down while rotating the outer housing 14. As shown in FIG. 10, an inner surface of the tab 76 can have a protrusion 78 formed thereon that is configured to engage the locking ring 28. The protrusion 78 can have any size and shape such as a longitudinally extending bar (as shown) that distally extends a distance beyond a distal surface of the outer housing's upper portion 16a. The locking ring 28 can have a corresponding depression 80 configured to receive the protrusion 78. The depression 80 can also have any size and shape, such as a rectangular channel (as shown). When the pins 72 are inserted into the slots 74 and rotated in the first direction relative thereto such that the pins 72 abut the terminal ends 74a of the slots 74, the protrusion 78 can automatically engage the depression 80, e.g., by moving radially outward to snap therein. The tab 76 can be held in the released position when the pins 72 are inserted into the slots 74 to help prevent the protrusion 78 from interfering with attachment of the outer housing 14 to the locking ring 28. To move the tab 76 from the engaged to the released position, the tab 76 can be pressed radially inward to move the protrusion radially inward and out of engagement from the depression 80.

With the housing 12 locked to the locking ring 28, e.g., with the pins 72 abutting the slot's terminal ends 74a and with the locking mechanism in the engaged position, the inner housing 16 can be rotated in the first direction, e.g., a clockwise direction, and in the second, opposite direction, e.g., a counter clockwise direction, to rotate the inner housing 16 relative to the outer housing 14 as well as to the locking ring 28 and to the retractor 18 when the locking ring 28 is attached thereto. As shown, the inner housing 16 can be configured to rotate 360°. While the inner housing 16 can be configured to be rotatable in only one of the first and second directions and/or less than 360°, the inner housing 16 as illustrated is rotatable 360° in both the first and second directions, which can help more effectively position surgical instruments inserted through the inner housing 16 with respect to each other. As mentioned above, the retractor protector 42 can rotate with the inner housing 16, thereby allowing the retractor protector 42 to maintain a consistent orientation relative to any surgical instruments inserted through the inner housing 16 and into the retractor 18.

Although the inner housing 16 can be configured to be movable relative to the outer housing 14 and the retractor 18 with or without any instruments inserted through any of the sealing ports 22a, 22b, 22c, e.g., by being manually rotated by hand, the inner housing 16 can also be configured to move relative to the outer housing 14 and the retractor 18 in response to motion of at least one instrument inserted through one of the ports 22a, 22b, 22c.

If disengagement of the outer housing 16 and the locking ring 28 is desired, e.g., to replace the housing 12 with another housing having a different number or different sizes of sealing ports or to replace the retractor 18, the outer housing 16 can be rotated in the second direction such that the bayonet pins 72 are free to be withdrawn from the slots 74. If the device 10 includes a locking mechanism, it can be moved from the engaged position to the released position, e.g., by depressing the tab 76, to allow rotation and removal of the outer housing 14 from the locking ring 28.

In use, one or more surgical instruments can be inserted into a body cavity through the surgical access device 10, which can help optimally position the surgical instruments relative to the body cavity through movement of the inner housing 16 relative to the retractor 18. The device 10 can be positioned within tissue to provide access to a body cavity underlying the tissue in a variety of ways. In one embodiment, the device 10 can be positioned in tissue fully assembled in the default position shown in FIGS. 1 and 3. In another embodiment, the device 10 can be positioned partially assembled in tissue and be fully assembled with a portion of the device 10 positioned in the tissue. The various elements of the device 10 can be attached together in any order. In one embodiment, the device 10 can be positioned in tissue by first positioning the retractor 18 therein, attaching the ring assembly 26 to the retractor 18 positioned in the tissue, and then attaching the housing 12 to the ring assembly 26.

The retractor 18 can be positioned within an opening or incision formed in tissue (generally referred to as an “opening”), e.g., in the umbilicus, with the proximal and distal flanges 29, 31 of the retractor 18 positioned on opposed sides of the tissue. The opening can have any shape and size, e.g., a linear cut having a longitudinal length in a range of about 15 to 35 mm and extending through a layer of tissue having a depth of less than about 70 mm. The retractor 18 can be positioned within a tissue opening in a variety of ways. In one embodiment, shown in FIGS. 11-14, the retractor 18 can be positioned in the opening by hand.

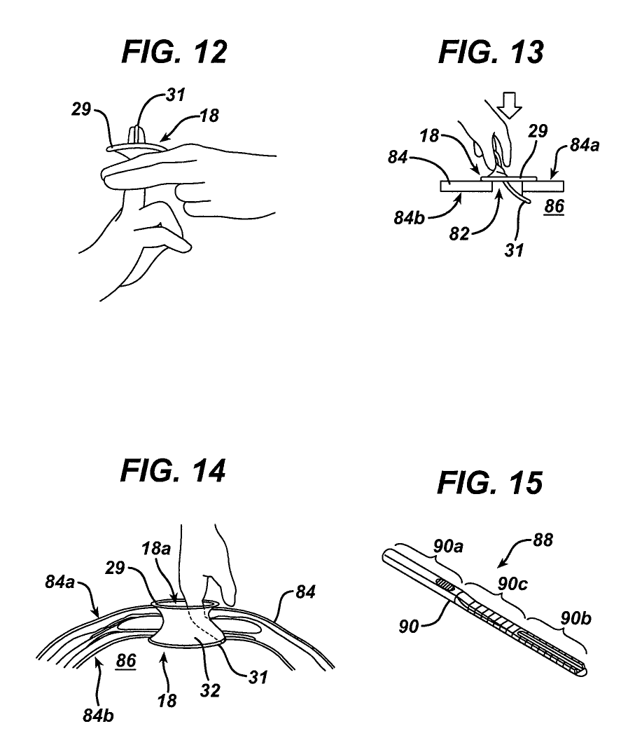

As shown in FIG. 11, while holding the proximal flange 29 of the retractor 18, the distal flange 31 can be pushed down or distally to flex the inner elongate portion 32 of the retractor 18. Then, as shown in FIG. 12, the distal flange 31 can be pushed up or proximally to push the distal flange 31 partially through the inner lumen 18a of the retractor 18 such that the distal flange 31 is in an angled position through the inner lumen 18a. Any amount of the distal flange 31 can be pushed through the retractor's inner lumen 18a, e.g., about one-third of the distal flange 31 above or proximal to the proximal flange 29. With the distal flange 31 in the angled position, the retractor 18 can be positioned in an opening 82 in tissue 84. As illustrated in FIG. 13, opposing the distal flange 31, a finger can be inserted distally through the retractor's inner lumen 18a. Grasping the proximal flange 29 and the portion of the distal flange 31 extending above the proximal flange 29, and with the proximal flange 29 positioned above or on a proximal surface 84a of the tissue 84 outside the patient, the angled-down portion of the distal flange 31 can be inserted through the tissue opening 82 and into a body cavity 86 underlying the tissue 84. When the distal flange 31 passes into the body cavity 86 and is positioned under or on a distal surface 84b of the tissue 84, a remainder of the distal flange 31 can be pushed through the opening 82 to be positioned in the body cavity 86 under or on the tissue's distal surface 84b. The retractor 18 can thus be positioned in the tissue opening 82, as illustrated in FIG. 14, with the proximal flange 29 of the retractor 18 positioned on and/or proximal to the proximal surface 84a of the tissue 84, and the distal flange 31 of the retractor 18 positioned on and/or distal to the distal surface 84b of the tissue 84 in the body cavity 86. The inner elongate portion 32 of the retractor 18 can thereby be positioned within the tissue 84 with the inner lumen 18a of the retractor 18 extending through the tissue to provide a path of access to the body cavity 86. Also as shown in FIG. 14, a finger can be inserted through the retractor's inner lumen 18a and swept along a margin between the distal flange 31 and the tissue's distal surface 84b to tactilely confirm that the distal flange 31 is positioned in the body cavity 86 against the tissue's distal surface 84b and not trapped in the tissue opening 82. The retractor's seating in the tissue 84 can alternatively or additionally be visually identified, e.g., using an endoscope.

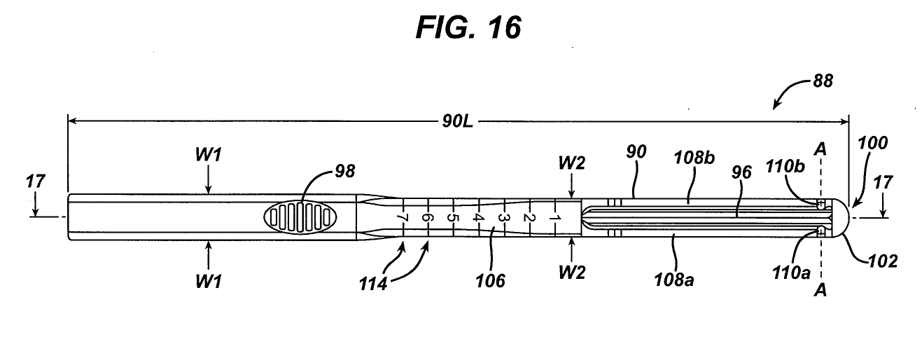

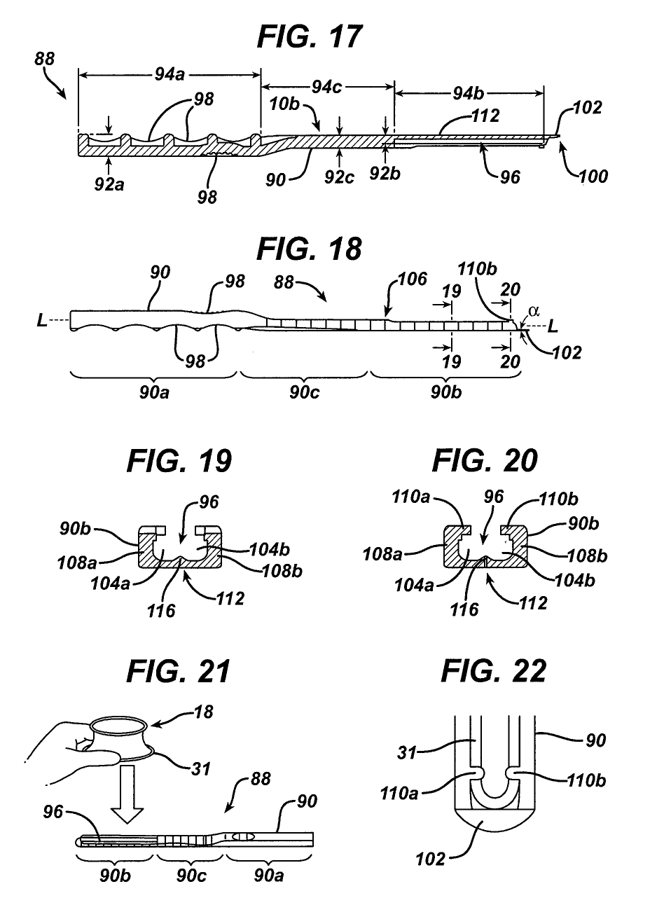

In another embodiment, shown in FIGS. 21-25, the retractor 18 can be positioned in an opening in tissue using an inserter tool 88, illustrated in FIGS. 15-20. Although the inserter tool 88 is shown in FIGS. 21-25 in use with the retractor 18 of the device 10 of FIGS. 1-3, a person skilled in the art will appreciate that the inserter tool 88 can be used with any flexible retractor configured to be positioned in an opening in tissue.

The inserter tool 88 can have a variety of sizes, shapes, and configurations. As shown in the embodiment illustrated in FIGS. 15-20, the inserter tool 88 can include an elongate shaft 90 having a proximal handle portion 90a, a distal retainer portion 90b, and a mid-portion 90c extending therebetween. Generally, the handle portion 90a can be configured to be held and manipulated outside a body of a patient while the retainer portion 90b and the mid-portion 90c can be configured to be at least partially inserted into the patient's body to position a retractor in a tissue opening. The retainer portion 90b can include a channel 96 extending longitudinally through at least a portion thereof, the channel 96 being configured to releasably couple to a retractor, as discussed further below.

The inserter tool 88 can be made from any combination of rigid and/or flexible materials, but in an exemplary embodiment the materials are biocompatible and suitable for use in surgical procedures. A person skilled in the art will appreciate that the term “flexible” as used herein is intended to encompass a variety of configurations. Generally, a “flexible” member is one which to at least some degree of elasticity is capable of bending or deforming without breaking. In an exemplary embodiment, the inserter tool 88 or at least portions thereof are composed of at least one biocompatible and flexible material, such as an elastomer, e.g., polyurethane, having a durometer in a range of about 35 to 65 Shore A, e.g., about 55 Shore A.

The inserter tool's shaft 90 can have any size, shape, and configuration, as will be appreciated by a person skilled in the art. The shaft 90 can be rigid, flexible, or a combination thereof, but in the illustrated embodiment it is flexible along its longitudinal length 90L. The handle portion 90a, the distal retainer portion 90b, and the mid-portion 90c can have varying degrees of flexibility, such as shown with the shaft 90 having a variable thickness. In an exemplary embodiment, the handle portion 90a has a largest thickness 92a and is the least flexible portion of the shaft 90, the retainer portion 90b has a smallest thickness 92b and is the most flexible portion of the shaft 90, and the mid-portion 90c has a thickness 92c and a flexibility between the terminal end portions 90a, 90b. By being formed of a relatively soft elastomer and having a relatively small thickness 92b, the retainer portion 90b can have enough structural integrity to be advanced through a tissue opening and be configured to flex without breaking and to dynamically return from a flexed position to a default or straight position, e.g., as shown in FIGS. 15-20.

The shaft 90 can vary in longitudinal length depending on the device's intended application. The proximal handle portion 90a, the distal retainer portion 90b, and the mid-portion 90c of the shaft 90 can also each have any respective longitudinal lengths 94a, 94b, 94c along the shaft 90 that added together equal the device's longitudinal length 90L, e.g., about 12.2 in. (31.0 cm). In an exemplary embodiment, the retainer portion 90b and the mid-portion 90c can each be about 25% of the tool's longitudinal length 90L, and the handle portion 90a can be about 50% of the tool's longitudinal length 90L.

The shaft 90 can be formed from a single component or multiple segments. The flexibility of the shaft 90, as well as its relatively small thickness, e.g., a maximum of about 0.55 in. (13.97 mm), can allow the tool 88 to be used in endoscopic procedures, whereby the tool 88 is introduced translumenally through a natural or artificial orifice. In an exemplary embodiment, the shaft 90 can have a substantially rectangular cross-section with rounded edges (see FIGS. 19 and 20), which can help ease the shaft's passage into an opening in tissue and prevent the shaft 90 from harming or getting caught on tissue.

The shaft 90 can have a uniform or non-uniform outer width along its longitudinal length 90L. In the illustrated embodiment, the handle portion 90a of the shaft 90 has a substantially uniform outer width W1 along its longitudinal length 94a while a remaining portion 90b, 90c of the shaft 90, e.g., a distal portion of the tool 88 configured to be at least partially inserted through tissue, has a smaller substantially constant width W2 along its longitudinal length 94b, 94c.

Generally, the retainer portion 90b of the tool 88 can be configured to mate to a retractor, to deliver the retractor through a tissue opening, and to dynamically or automatically release the retractor into position within the tissue opening. A distal insertion tip 102 can be located at a distal-most end 100 of the retainer portion 90b to help ease insertion of the retainer portion 90b into a body of a patient, such as by the distal tip 102 having a rounded tip as illustrated in this embodiment. The distal tip 102 can be substantially flat, or it can taper or slope any number of degrees, such as shown in FIG. 18 with an upward angle a of about 4°, to help facilitate distal advancement of the distal tip 102 through tissue. Opposed side rails 108a, 108b in the retainer portion 90b defining the channel 96 can taper down to the distal tip 102.

As mentioned above, the retainer portion 90b can include the channel 96 formed therein that can be configured to hold and release a retractor. The channel 96 can have any longitudinal length along the shaft 90 and can have a variety of shapes, size, and configurations. As shown in the illustrated embodiment, the channel 96 can extend to a proximal end of the retainer portion 90b from a location proximal to a distal-most end 100 of the shaft 90, e.g., between the distal tip 102 and the mid-portion 90c. In this way, a retractor held in the channel 96 can be prevented from sliding distally beyond the distal-most end 100 of the shaft 90. The channel 96 can have any shaped cross-section, e.g., a generally c-shaped cross-section (as shown), defined by the opposed side rails 108a, 108b and a lower surface 112 of the shaft 90 in the retainer portion 90b and having an opening extending through an upper surface 106 of the shaft 90. By having a substantially curved cross-sectional shape, the channel 96 can be less likely to damage a retractor disposed therein. As in the illustrated embodiment, the channel 96 can include first and second longitudinally extending recesses 104a, 104b separated by a longitudinally extending rib 116 extending upwards from the bottom surface 112 into the channel 96. The rib 116 can help provide structural integrity to the retainer portion 90b while allowing the bottom surface 112 of the shaft 90 between the rib 116 and the side rails 108a, 108b to be thin to aid in flexibility of the retainer portion 90b. The recesses 104a, 104b can each have generally c-shaped cross-sections, which can allow the recesses 104a, 104b to seat opposed portions of a flexible ring such as a proximal or distal flange of a retractor, e.g., the proximal flange 29 or the distal flange 31 of the retractor 18 of FIGS. 1-3. The proximal and distal flanges 29, 31 in the illustrated retractor embodiment are identical substantially circular rings having the flexible o-rings 29a, 31 a disposed therein, which can allow either of the flanges 29, 31 to be positioned in the channel 96 and either to form the distal portion of the retractor 18 disposed in a body cavity of a patient. Such interchangeability can help reduce delay during a surgical procedure. Although the channel 96 is configured to seat the one of the flanges 29, 31 in the illustrated embodiment such that the retractor 18 can extend outward from the shaft 90 when mated to the inserter tool 88, the channel 96 can be configured to seat any portion of a retractor.

To help retain a retractor in position within the channel 96, the inserter tool 88 can include one or more retention members configured to prevent the retractor from being prematurely released from the tool 88. As will be appreciated by a person skilled in the art, the retention members can have a variety of shapes, sizes, and configurations. In the illustrated embodiment, the inserter tool 88 includes first and second retention members 110a, 110b each extending adjacent to the shaft's upper surface 106 from one of the opposed side rails 108a, 108b and radially inward toward the channel 96. The retention members 110a, 110b, as shown, can be at the same axial position A along the shaft's longitudinal axis L and can each be in the form of identical, substantially flat, bullet-shaped protrusions having rounded distal tips. The retention members 110a, 110b can extend any distance from the side rails 108a, 108b and over the channel 96 such that the retention members 110a, 110b can be configured to prevent the portion of the retractor disposed in the channel 96 from being pulled laterally out of the channel 96, e.g., from being pulled through the channel's opening and away from the shaft's upper surface 106. In an exemplary embodiment the retention members' distal tips do not touch, thereby allowing the portion of the retractor retained in the channel 96 to be released from between the retention members 110a, 110b, as discussed further below.

Generally, the mid-portion 90c can be configured to facilitate insertion of the retainer portion 90b within a tissue opening by being at least partially insertable into the tissue opening. The mid-portion 90c can have a variety of sizes, shapes, and configurations. As shown in this embodiment, the mid-portion 90c can include indicia 114 printed, embossed, or otherwise visible thereon that is configured to indicate an insertion depth of the shaft 90 through an opening in tissue and into a body cavity. As will be appreciated by a person skilled in the art, the indicia 114 can have a variety of sizes, shapes, and configurations. In the illustrated embodiment, the indicia 114 includes a plurality of lines printed circumferentially around the shaft 90 that are perpendicular to the shaft's longitudinal axis L. The mid-portion 90c can include any number of lines, and the lines can be spaced apart any distance to indicate various degrees of insertion depth, e.g., seven lines spaced about 1 cm apart and numbered one to seven in a proximal direction.

Generally, the handle portion 90a can be configured to facilitate grasping of the tool 88. The handle portion 90a can have a variety of sizes, shapes, and configurations. Non-limiting examples of the handle portion 90a include an elongate rod (as shown), a finger loop, a knob, an enlarged grip, etc. The handle portion 90a can optionally include one or more hand or finger grips 98, e.g., an indented and/or textured surface, configured to help facilitate secure, non-slip grasping of the tool 88. The handle portion 90a can form a non-insertion section of the tool 88, e.g., a section of the tool 88 not configured to be inserted into a body of a patient, while a remainder of the tool 88, e.g., the mid-portion 90c and the retainer portion 90b, can form an insertion section of the tool 88, e.g., a section of the device 10 configured to be inserted at least partially into a body of a patient.

Although as mentioned above the inserter tool 88 can be used with any retractor, the tool 88 is illustrated in use in FIGS. 21-25 to position the retractor 18 of FIGS. 1-3 in the opening 82 formed in the tissue 84. To mate the retractor 18 to the tool 88, the retractor's distal flange 31 can be pinched together as shown in FIG. 21 from an expanded, default configuration (as shown in FIGS. 1-3) to a collapsed configuration and inserted into the channel 96 in the distal retainer portion 90b of the tool 88. The distal flange 31 can be positioned in the collapsed configuration under the retention members 110a, 110b as shown in FIG. 22 to help retain the retractor 18 in the tool 88. Also as shown in FIG. 22, each retention member 110a, 110b can radially extend inwards a distance larger than a cross-sectional diameter of the distal flange 31 to further help prevent the distal flange 31 from prematurely sliding out of the channel 96.

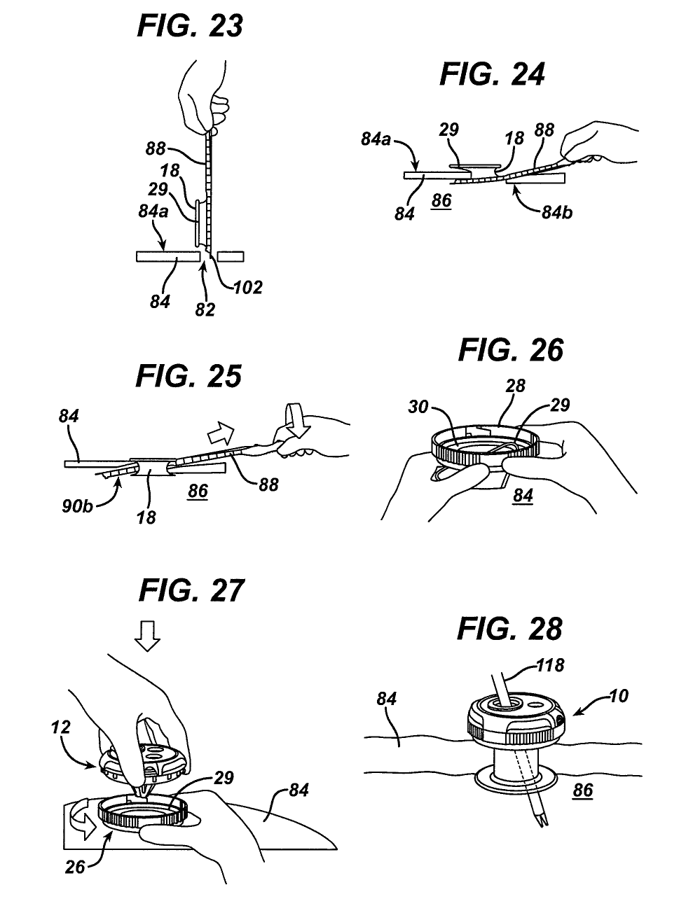

With the retractor 18 disposed in the channel 96 of the tool 88, the tool 88 and the retractor 18 can be inserted into the tissue opening 82, as shown in FIG. 23. As will be appreciated by a person skilled in the art, to facilitate installation of the retractor 18 in the tissue opening 82 using the inserter tool 88, a lubricant (not shown), e.g., water based surgical lubricant, can be applied to the inserter tool 88. The lubricant can be applied at any time during a surgical procedure, such as following mating of the retractor 18 to the tool 88 but prior to insertion of the tool 88 through the tissue 84. With the retractor 18 mated to a distal portion thereof, the inserter tool 88 can be inserted distal tip 102 first into the tissue opening 82 and advanced distally with light, controlled, downward pressure. The tool 88 can be held and inserted substantially perpendicular to the proximal surface 84a of the tissue 84, as illustrated in FIG. 23, which can help allow the tapered distal tip 102 to enter and dilate the tissue opening 82 and the proximal flange 29 of the retractor 18 to abut and remain outside the tissue's proximal surface 84a.

As the inserter tool's distal tip 102 and the retractor 18 enter the body cavity 86 underlying the tissue 84, as shown in FIG. 24, the inserter tool 88 can be angled to flex at least the retainer portion 90b of the shaft 90 and direct at least the retainer portion 90b of the shaft 90 in a direction parallel to the body cavity 86 and the inner or distal surface 84b of the tissue 84, which can help prevent the tool 88 and/or the retractor 18 from interfering with vessels or other anatomic structures underlying the tissue 84. Such angling can also help direct the proximal flange 29 of the retractor 18 to engage and be substantially parallel to the tissue's proximal surface 84a. The indicia 114 on the inserter tool 88 can help indicate an insertion depth of the tool 88 inserted through the tissue opening 82, which can help indicate when the retractor 18 has entered the body cavity 86. For non-limiting example, if the tissue opening 82 is about 4 cm deep and the line marked “4” is substantially at the outer or proximal surface 84a of the tissue 84, then the retractor 18 has likely been inserted into the body cavity 86. With the proximal flange 29 of the retractor 18 engaging the tissue's proximal surface 84a, flexing the retainer portion 90b can allow release of the distal flange 31 of the retractor 18 from the tool 88. When the retainer portion 90b is flexed enough for the retractor 18 held therein to apply a predetermined force to the channel 96 that exceeds a force holding the proximal flange 29 of the retractor 18 outside the tissue 84, the side rails 108a, 108b can radially move apart from one another such that the channel 96 can widen in at least a portion thereof and the retention members 110a, 110b can radially move apart from one another, thereby allowing the channel 96 to release the retractor 18 into position within the tissue opening 82, as shown in FIG. 25. The predetermined force can vary and can generally be based on the material used to form the tool's retainer portion 90b and on the thickness 92b of the retainer portion 90b. Release of the retractor 18 from the tool 88 can be felt through the handle portion 90c held outside the body. With the distal flange 31 released from the tool 88, the retractor 18 can be positioned within the tissue opening 82 as shown in FIG. 25 with the proximal and distal flanges 29, 31 positioned on opposite sides of the tissue 84. With the retainer portion 90b being formed of a relative soft elastomer and having the retractor 18 released therefrom, the retainer portion 90b can relatively slowly flex back to its default position rather than harshly snapping back into the default position and potentially damaging the tissue 84 or other anatomical structure.

To remove the tool 88 from the body, the tool 88 can be rotated clockwise and/or counter-clockwise in any amount, e.g., about 180°, and withdrawn from the tissue opening 82 as also shown in FIG. 25. The retractor 18 can thus be positioned in the tissue opening 82, as illustrated in FIG. 14, with the proximal flange 29 of the retractor 18 positioned on and/or proximal to the proximal surface 84a of the tissue 84, and the distal flange 31 of the retractor 18 positioned on and/or distal to the distal surface 84b of the tissue 84 in the body cavity 86. The inner elongate portion 32 of the retractor 18 can thereby be positioned within the tissue 84 with the inner lumen 18a of the retractor 18 extending through the tissue 84 to provide a path of access to the body cavity 86. Also as shown in FIG. 14, a finger can be inserted through the retractor's inner lumen 18a and swept along a margin between the distal flange 31 and the tissue's distal surface 84b to tactilely confirm that the distal flange 31 is positioned in the body cavity 86 against the tissue's distal surface 84b and not trapped in the tissue opening 82. The retractor's seating in the tissue 84 can alternatively or additionally be visually identified, e.g., using an endoscope.

With the retractor 18 positioned in the tissue 84, either by hand or through use of the inserter tool 88, the ring assembly 26 and the housing 12 can be attached to the retractor 18. Although, as mentioned above, in some embodiments the ring assembly 26 with or without the housing 12 attached thereto can be mated to the retractor 18 when the retractor 18 is positioned in a tissue opening. In an exemplary embodiment, as shown in FIG. 26, the locking ring 28 and the retractor ring 30 can be attached together, and then the retractor ring 30 can be mated to the proximal flange 29 of the retractor 18 positioned outside the body. If the tissue 84 and/or the retractor 18 are adequately flexible, the retractor 18 can be angled or pivoted to a desired position to ease attachment of the retractor ring 30 to the retractor 18. The retractor 18 can also be angled or pivoted during use of the device 10 with one or more surgical instruments inserted therethrough. To mate the retractor ring 30 and the retractor 18, the retractor's proximal flange 29 can be pulled by hand and/or with one or more surgical instruments through an inner lumen of the retractor ring 30 to position the proximal flange 29 on an inner circumferential lip 30a of the retractor ring 30 (see FIGS. 3 and 9). The inner circumferential lip 30a can continuously run circumferentially around the retractor ring 30 as shown, or the inner circumferential lip 30a can run around one or more discrete portions of the retractor ring 30. The inner circumferential lip 30a of the retractor ring 30 can optionally include a beveled surface, which can help secure the retractor 18 to the proximal portion 24 of the device 10 as discussed further below. The retractor ring 30 can thus have the proximal flange 29 disposed therein to couple the retractor 18 and the ring assembly 26 together.

With the retractor 18 positioned in the tissue 84 and having the ring assembly 26 attached thereto, the housing 12 can be attached to the ring assembly 26 and the retractor 18 as shown in FIG. 27 to fully assemble the device 10. Although, as mentioned above, in some embodiments the housing 12 can be attached to the ring assembly 26 when the ring assembly 26 is mated to the retractor 18. To mate the housing 12 to the ring assembly 26 and the retractor 18, the housing 12 can be positioned proximal to the ring assembly 26 and the retractor 18 with a distal portion of the housing 12 engaging a proximal portion of the ring assembly 26 and the retractor 18, e.g., a distal portion of the housing 12 engaging the locking ring 28. As mentioned above, the bayonet pins 72 of the housing 12 can be positioned in the slots 74 of the locking ring 28, and the housing 12 can be rotated relative to the locking ring 28 to lock the housing 12 thereto. The tissue 84 can provide adequate tension such that the locking ring 28, attached to the retractor 18 positioned in the tissue 84, need not be held in position while the housing 12 is rotated relative thereto, although the ring assembly 26 and/or the retractor 18 can be so held to help provide support to the device 10 during its assembly.

The outer housing 14 and the retractor ring 30 can each include a beveled surface configured to engage the proximal flange 29 of the retractor 18 therebetween. The beveled surfaces can help accommodate an uneven surface of the proximal flange 29 that can result from a bonded edge created during manufacture of the proximal flange 29, e.g., when the proximal o-ring 29a is positioned therein as discussed above. The bonded edge can have an uneven size and/or shape on a single retractor and can also vary in size and/or shape between individual retractors. The beveled surfaces can thus improve the compatibility of the proximal portion 24 of the device 10 with a variety of retractors. As shown in FIGS. 3, 5, and 7, the lower portion 14b of the outer housing 14 can include a beveled distal surface of the distal lip 14c configured to cooperate with a beveled proximal surface of the inner circumferential lip 30a of the retractor ring 30. The beveled distal surface of the distal lip 14c and the beveled proximal surface of the inner circumferential lip 30a can each angle radially inward and proximally upward as illustrated to help hold the proximal flange 29 therebetween and provide adequate space for the flange's bonded edge.

With the surgical access device 10 assembled and positioned in the tissue 84, as shown in FIG. 28, one or more surgical instruments 118 can be inserted therethrough through any one or more of the sealing ports 22a, 22b, 22c and into the body cavity 86 where the instruments 118 can help perform any type of surgical procedure. Prior to insertion of the instrument 118 through the device 10, insufflation can be provided using the insufflation port 34, the tubing 36, and the stopcock 38. Although the surgical instrument 118 shown in FIG. 28 is a grasper having a pair of distal movable jaws, a person skilled in the art will appreciate that any surgical instrument can be inserted through the device 10. Further, although the instrument 118 is illustrated as being inserted through the larger sealing port 22a with the reducer cap 52 attached thereto, as mentioned above, an instrument can be inserted through the larger sealing port 22a with the reducer cap 52 removed therefrom.

At any point before, during, or after a surgical procedure, part of all of the proximal portion 24 of the device 10 can be released from the retractor 18, and the retractor 18 can be removed from the tissue. To disengage the housing 12 from the ring assembly 26 and the retractor 18, the housing 12 can be rotated relative to the ring assembly 26 and the retractor 18. As mentioned above, the tab 76 can be depressed to allow such rotation. The engagement and release mechanism can then be disengaged, e.g., the housing 12 can be proximally moved to disengage the bayonet pins 72 from the openings 74. The tissue 84 can provide adequate tension for the proximal motion of the housing 12.

With the housing 12 of the device 10 disengaged from the ring assembly 26 and the retractor 18, the working channel 18a of the retractor 18 can still provide access to the body cavity 86 underlying the tissue 84. With or without any or all of the ring assembly 26 removed from the retractor 18, one or more surgical instruments can be advanced through the working channel 18a, such as a waste removal bag configured to hold waste material, e.g., dissected tissue, excess fluid, etc., from the body cavity 86. The bag can be introduced into the body cavity 86 through the retractor's working channel 18a or other access port. A person skilled in the art will appreciate that one or more surgical instruments can be advanced through the retractor's working channel 18a before and/or after the housing 12 and/or the ring assembly 26 has been attached to the retractor 18.

The retractor 18 can be removed from within the tissue opening 82 in any way. In some embodiments, the retractor 18 can be pulled out of the opening 82 by hand, e.g., by inserting a finger through the retractor's inner lumen 18a and pulling the distal flange 31 from the body cavity 86 through the proximal flange 29. In some embodiments, a string, thread, suture, or cord (generally referred to as a “cord”) can be used to help remove the retractor 18 from the tissue 84, with or without the housing 12 and/or the ring assembly 26 being attached to the retractor 18. The cord can have a variety of sizes, shapes, and configurations. Generally, the cord can be a surgically safe flexible material, such as umbilical tape.

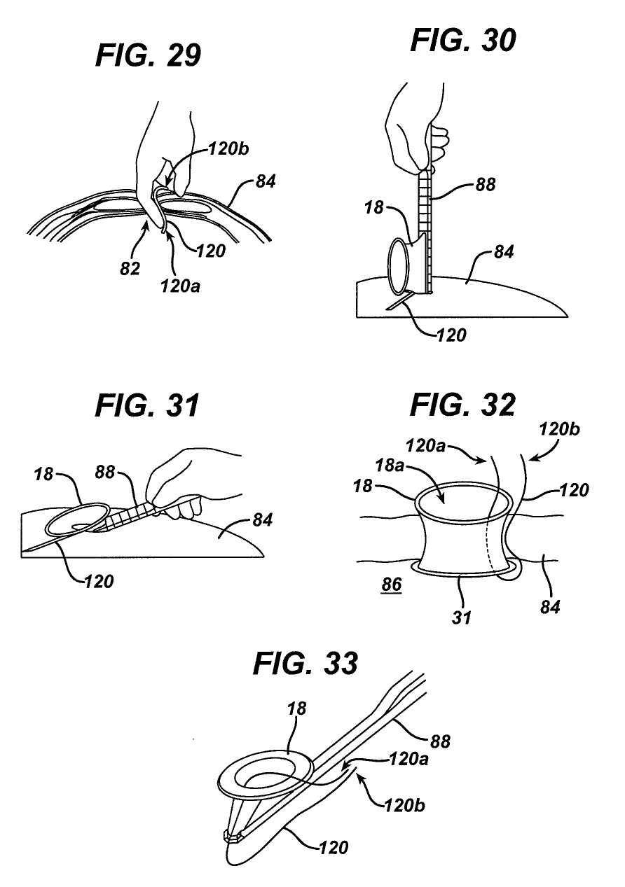

The cord can be coupled to the retractor 18 before or after the retractor is positioned in the tissue opening 82. In one embodiment shown in FIGS. 29-31, a cord 120 can be positioned in the tissue opening 82 before placement of the retractor 18 therein. Although the retractor 18 is shown being inserted into the tissue 84 in FIGS. 30 and 31 using the inserter tool 88, the retractor 18 can be positioned in the tissue 84 by hand as discussed above. Further, also as discussed above, the retractor 18 can be positioned in the tissue 84 with any part of the device's proximal portion 24 attached thereto. In use, as shown in FIG. 29, the cord 120 can be inserted into the tissue opening 82 such that the cord 120 is in an extended configuration with a first terminal end 120a of the cord 120 disposed in the body cavity 86 and a second terminal end 120b of the cord 120 located outside the body of the patient. With the cord 120 in the extended configuration, the retractor 18 can be positioned in the tissue opening 82 using the inserter tool 88 as discussed above and as illustrated in FIGS. 30 and 31. The inserter tool 88 can be removed from the body, and a length of the cord 120 can be positioned between the tissue 84 and the retractor 18 in the tissue opening 82 with the cord's first and second terminal ends 120a, 120b remaining on opposite sides of the tissue 84. The first terminal end 120a of the cord 120 can be pulled through the retractor's working channel 18a by hand and/or with at least one surgical instrument from inside the body cavity 86 to a location outside the body of the patient, such that the cord 120 can be in a looped configuration around the retractor 18 as shown in FIG. 32 with the cord 120 extending through the retractor's working channel 18a, around the distal flange 31, and between the retractor's flexible inner elongate portion 32 and the tissue 84. Thus, while the cord 120 can have any longitudinal length, in an exemplary embodiment the cord's longitudinal length can be at least twice a height of the retractor 18.

In another embodiment shown in FIG. 33, the cord 120 can be pre-threaded through the retractor 18 in the looped configuration, before or after the retractor 18 is mated to the inserter tool 88. The inserter tool 88 can then be used to position the retractor 18 in the tissue opening 82 as discussed above to position the retractor 18 and the cord 120 as shown in FIG. 32.

With the cord 120 in the expanded or looped configuration and with the retractor 18 positioned in the tissue 84, the ring assembly 26 and the housing 12 can be attached to the retractor 18 as discussed above to fully assemble the device 10. While the cord 120 can be positioned anywhere with the proximal portion 24 of the device 10 mated to the retractor 18, in one embodiment with the cord 120 in the looped configuration, the cord 120 can be positioned between the retractor's proximal flange 29 and the lower portion 14b of the outer housing 14 with the terminal ends 120a, 120b of the cord 120 extending outside the device 10. The cord 120 can be tensioned prior to full assembly of the device 10 to help minimize a length of the cord 120 extending through the retractor's working channel 18a. In another embodiment with the cord 120 in the expanded configuration, the proximal portion 24 of the device 10 can be mated to the retractor 18 with the first terminal end 120a of the cord 120 located in the body cavity 86.

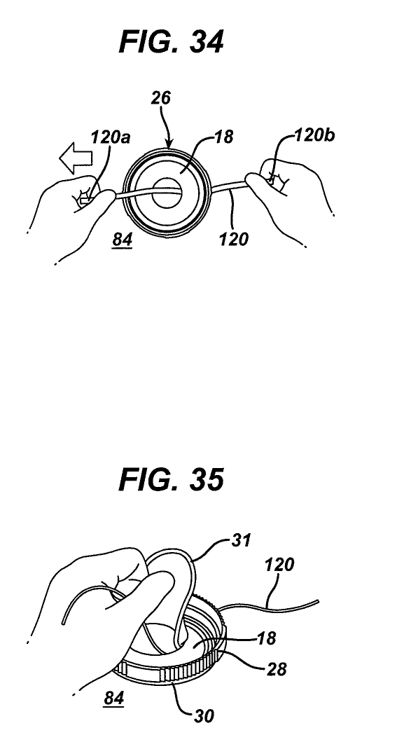

At any point during the surgical procedure, the cord 120 can be used to remove the retractor 18 from the tissue 84. If both terminal ends 120a, 120b are not located outside the body, part or all of the proximal portion 24 of the device 10 can be detached from the retractor 18 to access and position both terminal ends 120a, 120b outside the body. As shown in one embodiment in FIGS. 34 and 35, the retractor 18 can be removed from the tissue 84 using the cord 120 with the ring assembly 26 attached to the retractor 18. Holding the terminal ends 120a, 120b of the cord 120, the first terminal end 120a of the cord 120 can be pulled to proximally move the distal flange 31 through the working channel 18a of the retractor 18 and out of the body of the patient. Once accessible, the distal flange 31 can be grasped, in addition to or in alternative to the cord's first terminal ends 120a, and pulled in a proximal direction to remove the retractor 18 from the tissue 84. In this way, with the cord 120 prepositioned between the retractor 18 and the tissue 84, no fingers or surgical instruments need be introduced any distance into the body to remove the retractor 18 from the tissue 84.

A surgical drape can optionally be placed over the retractor 18 and the tissue opening 82 during removal of the retractor 18 to help reduce dispersion of bodily fluid outside the surgical space.

As will be appreciated by those skilled in the art, any and all of the embodiments disclosed herein can be interchangeable with one another as needed. For example, an exemplary surgical access device kit could include multiple housings with one or more retractors. Each housing can have different sealing port configurations including different types and numbers of sealing elements, etc. as needed in particular application. Various release mechanism known in the art can be used to releasably attach the various housings to a retractor.

There are various features that can optionally be included with any and all of the surgical access device embodiments disclosed herein. For example, a component of the device, such as an outer housing, retractor, sealing element, etc., can have one or more lights formed thereon or around a circumference thereof to enable better visualization when inserted within a patient. As will be appreciated, any wavelength of light can be used for various applications, whether visible or invisible. Any number of ports can also be included on and/or through the surgical access devices to enable the use of various surgical techniques and devices as needed in a particular procedure. For example, openings and ports can allow for the introduction of pressurized gases, vacuum systems, energy sources such as radiofrequency and ultrasound, irrigation, imaging, etc. As will be appreciated by those skilled in the art, any of these techniques and devices can be removably attachable to the surgical access device and can be exchanged and manipulated as needed.

The embodiments described herein can be used in any known and future surgical procedures and methods, as will be appreciated by those skilled in the art. For example, any of the embodiments described herein can be used in performing a sleeve gastrectomy and/or a gastroplasty, as described in U.S. application Ser. No. 12/242,765 entitled “Surgical Access Device” filed on Sep. 30, 2008; U.S. application Ser. No. 12/242,711 entitled “Surgical Access Device with Protective Element” filed on Sep. 30, 2008; U.S. application Ser. No. 12/242,721 entitled “Multiple Port Surgical Access Device” filed on September 30, 2008; U.S. application Ser. No. 12/242,726 entitled “Variable Surgical Access Device” filed on September 30, 2008; U.S. application Ser. No. 12/242,333 entitled “Methods and Devices for Performing Gastrectomies and Gastroplasties” filed on Sep. 30, 2008; U.S. application Ser. No. 12/242,353 entitled “Methods and Devices for Performing Gastrectomies and Gastroplasties” filed on Sep. 30, 2008; and U.S. application Ser. No. 12/242,381 entitled “Methods and Devices for Performing Gastroplasties Using a Multiple Port Access Device” filed on Sep. 30, 2008, all of which are hereby incorporated by reference in their entireties.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination, e.g., a housing, a retractor ring, a reducer cap, a distal retainer portion of an inserter tool, etc. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam, and a liquid bath (e.g., cold soak).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. An inserter tool, comprising:
an elongate shaft having a proximal handle portion and a flexible distal portion, the flexible distal portion having opposed side rails that define a channel extending longitudinally through at least a portion of the flexible distal portion, the channel including first and second longitudinally extending recesses configured to seat opposed portions of a flexible ring, and the flexible distal portion including first and second retention members extending from the opposed side rails and toward the channel such that the retention members are configured to prevent the flexible ring from being pulled laterally out of the channel.

2. The tool of claim 1, wherein the flexible distal portion has a thickness that is less than a thickness of the proximal handle portion.

3. The tool of claim 1, further comprising indicia on the elongate shaft configured to indicate an insertion depth of the elongate shaft through an opening in tissue and into a body cavity.

4. A surgical kit, comprising:
a cannula having proximal and distal flexible annular rings, and a flexible sidewall extending between the proximal and distal flexible annular rings and defining an inner lumen extending through the cannula; and
an inserter tool having a handle and a flexible elongate shaft extending from the handle, the flexible elongate shaft including a channel extending longitudinally therethrough and configured to seat one of the proximal and distal flexible annular rings to retain the seated annular ring in a collapsed configuration.

5. The kit of claim 4, further comprising a proximal assembly configured to mate to the proximal flexible annular ring and including a plurality of sealing ports each configured to receive an instrument inserted therethrough and into the inner lumen extending through the cannula.

6. The kit of claim 5, wherein the proximal assembly comprises a retractor ring releasably matable to the proximal flexible annular ring;
a locking ring releasably matable to the retractor ring in a fixed position; and
a housing releasably matable to the locking ring in a fixed position.

7. The kit of claim 6, wherein the housing comprises an outer housing releasably matable to the locking ring in a fixed position, the outer housing having an inner housing rotatably disposed therein, the inner housing having the plurality of sealing ports.

8. A surgical method, comprising:
positioning a distal annular ring on a cannula within a channel extending longitudinally through a flexible distal portion of an insertion tool such that the channel retains the distal annular ring in a collapsed configuration, the distal annular ring being coupled to a proximal annular ring by a flexible sidewall defining an inner lumen extending between the proximal and distal annular rings; and
advancing the flexible distal portion of the insertion tool through tissue such that the proximal annular ring on the cannula abuts an outer surface of the tissue and causes the flexible distal portion to flex, the channel releasing the distal annular ring when a predetermined force is applied to the channel.

9. The method of claim 8, wherein at least one retention member on the flexible distal portion extending toward the channel moves away from the channel when the predetermined force is applied to the channel to release the distal annular ring from the channel.

10. The method of claim 8, further comprising attaching a proximal assembly to the proximal annular ring when the proximal annular ring on the cannula abuts the outer surface of the tissue.

11. The method of claim 10, further comprising inserting an instrument through one of a plurality of sealing ports in the proximal assembly and through the inner lumen of the cannula to position a distal portion of the instrument in a body cavity underlying the tissue.

12. The method of claim 8, further comprising pulling a cord extending through the inner lumen, around the distal annular ring, and between the flexible sidewall and the tissue to pull the distal annular ring through the tissue to remove the cannula from the tissue.

13. A surgical method, comprising:
implanting a cannula through an incision in tissue to position a proximal annular ring adjacent to an outer surface of the tissue and to position a distal annular ring adjacent to an inner surface of the tissue such that a flexible sidewall extending between the proximal and distal annular rings forms an opening through the tissue, the cannula having a cord extending through the opening, around the distal annular ring, and between the flexible sidewall and the tissue; and
pulling the cord to pull the distal annular ring through the tissue to remove the cannula from the tissue.
